SEQUENCE LISTING (15 sequences)

SEQ ID NO: 1 - DNA, 2850 bp, Human adenovirus
atggccaccccatcgatgctgccccagtgggcatacatgcacatcgccggacaggatgcttcggagtacctgagtccgggtctggtgcagttcgcccgcgccacagacacctacttcaatctggggaacaagtttaggaaccccaccgtggcgcccacccatgatgtgaccaccgaccgcagtcagcggctgatgctgcgctttgtgcccgttgaccgggaagacaatacctacgcatacaaagttcgatacaccttggctgtgggcgacaacagagtgctggatatggccagcactttctttgacattcggggtgtgttggatagaggccctagcttcaagccatactctggcactgcttacaactcgttggcccctaagggcgctcccaatacatctcagtggattgctgaaggcgtaaaaaaagaagatgggggatctgacgaagaggaagagaaaaatctcaccacttacacttttggaaatgccccagtgaaagcagaaggtggtgatatcactaaagacaaaggtcttccaattggttcagaaattacagacggcgaagccaaaccaatttatgcagataaactataccaaccagaacctcaggtgggagatgaaacttggactgacacagatggaacaactgagaagtatggtggtagagctctaaagccagaaactaaaatgaaaccctgctatgggtcttttgctaaacccactaacgtcaaaggcggacaggcaaaacaaaaaactactgaacaaccgcaaaaccagcaggttgaatatgatattgacatgaacttttttgatgaagcgtcacagaaagcaaacttcagtccaaaaattgtgatgtatgcagaaaatgtagacttggaaaccccagacactcatgtggtgtacaaacctggtacttcagaagaaagttctcatgctaatctgggtcaacaatctatgcccaacagacccaactacattggctttagagataactttattggacttatgtactacaacagtactggcaacatgggagtgctggcaggtcaagcatcccaattgaatgcggtggttgacttgcaggacagaaacacagaactatcatatcaactactgcttgactctctgggtgacagaaccagatacttcagcatgtggaatcaagcagtcgatagctatgatcctgatgtgcgcattattgaaaatcatggggtggaagatgagcttcccaactactgctttccattggatggagtaggggtaccaataagtagttacaaaataattgaaccaaacggacagggtgcagattggaaagagcctgacataaatggaacaagtgaaattggacaaggaaatctctttgccatggaaattaacctccaagctaatctctggagaagttttctttattccaatgtggctctgtatctcccagactcctacaaatacaccccagccaatgtcactcttccaactaacaccaacacttatgactacatgaatgggcgggtggttcccccatccctggtggatacctacgtaaacattggcgccagatggtctttggatgccatggacaatgtcaacccctttaaccatcaccgcaacgctggcctgcgataccggtccatgcttttgggcaatggtcgttacgtgcctttccacattcaagtgcctcagaaattctttgctgtgaagaacctgctgcttctacccggttcttacacctacgagtggaacttcagaaaggatgtgaacatggtcctgcagagttcccttggtaatgatctccgggtcgatggtgccagcataagttttaccagcatcaatctctatgccaccttcttccccatggcccacaacactgcctccacccttgaagccatgctgcgcaatgacaccaatgatcaatcattcaatgactacctttctgctgccaacatgctctaccccatcccggccaacgctaccaacgttcccatctccattccctctcgcaactgggccgccttcagaggctggtccttcaccagactcaaaaccaaggagactccctctttgggatcagggttcgatccctactttgtttactctggttctataccctacctggatggtaccttctaccttaaccacactttcaagaaagtctccatcatgtttgactcttcagtgagctggcctggtaatgacagattgctaagtccaaatgagttcgaaatcaagcgcacagttgatggggaaggctacaatgtggcccaatgtaacatgaccaaagactggttcctggtccagatgcttgccaactacaacattggataccagggcttctacgttcctgagggttacaaggatcgcatgtactccttcttcagaaacttccagcccatgagtagacaggtggttgatgagattaactacaaagactataaagctgtcgccgtaccctaccagcataataactctggctttgtgggttacatggctcctaccatgcgtcagggtcaagcgtaccctgctaactacccataccccctaattggaaccactgcagtaaccagtgtcacccagaaaaaattcctgtgcgacaggaccatgtggcgcatcccattctctagcaacttcatgtccatgggtgcccttacagacctgggacagaacttgctgtatgccaactcggcccatgcgctggacatgacttttgaggtggatcccatggatgagcccaccctgctttatcttcttttcgaagtcttcgacgtggtcagagtgcaccagccacaccgcggcgtcatcgaggccgtctacctgcgcacaccgttctccgccggcaacgccaccacataa

SEQ ID NO: 2 - DNA, 32 bp, Artificial Sequence
  Description of Artificial Sequence Synthetic primer
ctgatgtactacaacagcactggcaacatggg

SEQ ID NO: 3 - DNA, 30 bp, Artificial Sequence
  Description of Artificial Sequence Synthetic primer
cggtggtggttaaatggattcacattgtcc

SEQ ID NO: 4 - DNA, 20 bp, Artificial Sequence
  Description of Artificial Sequence Synthetic primer
cgcccaatacatctcagtgg

SEQ ID NO: 5 - DNA, 20 bp, Artificial Sequence
  Description of Artificial Sequence Synthetic primer
actccaacttgaggctctgg

SEQ ID NO: 6 - DNA, 23 bp, Artificial Sequence
  Description of Artificial Sequence Synthetic probe
gttaaaaccgatgacactaatgg

SEQ ID NO: 7 - DNA, 24 bp, Artificial Sequence
  Description of Artificial Sequence Synthetic probe
ggtgtgggattgacagacacttac

SEQ ID NO: 8 - DNA, 23 bp, Artificial Sequence
  Description of Artificial Sequence Synthetic probe
gtggatagttacaacgggagaag

SEQ ID NO: 9 - DNA, 25 bp, Artificial Sequence
  Description of Artificial Sequence Synthetic probe
agaccaagcttggaaagatgtaaat

SEQ ID NO: 10 - DNA, 20 bp, Artificial Sequence
  Description of Artificial Sequence Synthetic probe
gggtgcagattggaaagagc

SEQ ID NO: 11 - DNA, 21 bp, Artificial Sequence
  Description of Artificial Sequence Synthetic probe
caaacggaaacggtaatcctc

SEQ ID NO: 12 - DNA, 20 bp, Artificial Sequence
  Description of Artificial Sequence Synthetic probe
ctaatggcaatggctcaggc

SEQ ID NO: 13 - DNA, 25 bp, Artificial Sequence
  Description of Artificial Sequence Synthetic probe
aaggtaaaacctaaaacaggtcagg

SEQ ID NO: 14 - DNA, 19 bp, Artificial Sequence
  Description of Artificial Sequence Synthetic probe
gctgctaacggggaccaag

SEQ ID NO: 15 - DNA, 19 bp, Artificial Sequence
  Description of Artificial Sequence Synthetic probe
aacaagcgagtggtggctc

(19) United States
(12) Patent Application Publication
(10) Pub. No.: US 2012/0264641 A1
(43) Pub. Date: Oct. 18, 2012

(54) METHODS AND KITS FOR IDENTIFYING HUMAN ADENOVIRUS SEROTYPES

(75) Inventor: Cicely Washington, Irvine, CA (US)

(73) Assignee: The Henry M. Jackson Foundation for the Advancement of Military Medicine, Inc., Bethesda, MD (US)

(21) Appl. No.: 13/514,710

(22) PCT Filed: Dec. 20, 2010
(86) PCT No.: PCT/US10/61256

§ 371 Date: Jun. 26, 2012

Related U.S. Application Data
(60) Provisional application No. 61/289,458, filed on Dec. 23, 2009.

(51) Int. Cl. C40B 30/04; C07H 21/04; C40B 40/06

(52) U.S. Cl. 506/9; 506/16; 536/24.32

(57) ABSTRACT

Methods, kits, primers and oligonucleotide probes for conveniently and rapidly detecting and identifying four or more human adenovirus (HAdV) serotypes in a sample are provided. Following a nucleic acid amplification reaction with specific primers, serotype specific oligonucleotide probes are used not only to detect HAdV present in a sample but also to discriminate between the HAdV serotypes present in the sample, and in particular to discriminate between the clinically relevant serotypes HAdV-3, HAdV-4, HAdV-7, HAdV-14, and HAdV-21 and/or HAdV-I, HAdV-2, HAdV-5, and HAdV-6. The combination of these primers and oligonucleotide probes permit the rapid and convenient serotyping of HAdV.

### CROSS REFERENCE TO RELATED APPLICATIONS

This application claims the benefit of, and relies on the filing date of, U.S. provisional patent application No. 61/289,458, filed 23 Dec. 2009, the entire disclosure of which is incorporated herein by reference.

### GOVERNMENT INTEREST

This invention was made in part with Government support under W81XWH-06-C-0414 awarded by USAMRAA. The Government may have certain rights in this invention.

### BACKGROUND

Human adenoviruses (HAdVs) have been known to cause a wide range of disease in humans, including upper and lower respiratory illness, urinary tract infections, conjunctivitis, and gastroenteritis (10, 12). There are 51 different HAdV serotypes based on type-specific serum neutralization, which are classified into six species (A, B, C, D, E, and F) on the basis of hemagglutination, oncogenic, and phylogenetic properties (3, 4, 9, 24, 25).

The most common serotypes known to cause respiratory illness in the adult population are 3, 4, 7, 11, 14 and 21 (11) (14) (15). All of these can cause locally severe outbreaks with high attack rates. These types of outbreaks are rarely reported in civilian populations, but are essentially continuous at military training camps, particularly with serotypes 4 and, to a lesser extent, 7. Some recent studies have suggested that specific serotypes cause more severe disease, especially in immunocompromised patients (7, 8, 11, 14, 17). Species C serotypes (1, 2, 5, and 6) are found predominately in children (20). The military previously established systematic vaccination for the two serotypes most commonly associated with training camp outbreaks, HAdV-4 and -7, which reduced adenovirus-induced ARD by 95%-99% (10). Production of these vaccines ceased in 1996, but similar replacement vaccines are currently in the final stages of licensure.

It is important to be able to identify the serotype and mutations in the serotype over time in order to evaluate viral virulence and vaccine and antiviral effectiveness. Identification of serotypes was traditionally done by virus isolation in cell culture (4), followed by neutralization tests, antibody studies and or antigen detection by immunofluorescence. These techniques are time consuming and labor intensive and subject to significant reciprocal cross reactions that can produce inaccurate stereotyping results (6, 8, 21). Another technique that has been used is whole genome restriction enzyme analysis (REA, or genome typing) which relies on large-scale viral culture to generate the full genomic substrate (2, 3).

More recently, PCR-based detection and discrimination methods have been developed (1, 11, 22, 23). These techniques are faster and can also detect co-infections when used in a multiplex assay (1, 11, 19, 23), thus reducing cost, labor and sample volume needed for analysis. While these PCR methods permit the rapid detection of HAdV in a sample, they typically require an additional sequencing, restriction enzyme, and/or phylogenetic analysis to discriminate between HAdV serotypes and, thus, significantly delay serotype identification.

For example, Xu et al. developed a multiplex PCR for identifying the six HAdV species A to F (23). Xu et al. designed six pairs of primers from the fiber gene, with each primer pair yielding a uniquely sized amplicon that was conserved within but variable between HAdV species. Thus, the primers of Xu et al. were capable of distinguishing among the HAdV species but could not discriminate between HAdV serotypes. Gu et al. developed a multiplex, real-time PCR assay for quantitatively detecting a wide range of HAdV serotypes (7). Using five primers (63 primer sequences including degenerate positions) and seven probes designed from the HAdV hexon gene, Gu et al. were able to detect all serotypes from species A, B, C, and E as well as eight serotypes from species D. However, the primers and probes used by Gu et al. were not capable of discriminating between HAdV serotypes and thus cannot be used to identify which HAdV serotypes may be present in a sample. Similarly, Wong et al. developed a multiplex real-time PCR assay capable of detecting HAdV from each of species A, B, C, D, and E (20). However the primers and probes of Wong et al. were not capable of discriminating between HAdV serotypes. Rather, Wong et al. used a separate sequencing reaction and phylogenetic analysis to determine HAdV serotype. Chmielewicz et al. identified a highly conserved 21 nucleotide region in the HAdV DPol gene (DNA polymerase) and used this sequence to design five primers and two probes for use in a PCR-based detection assay (4). This combination of primers and probes was capable of detecting all known HAdV serotypes. Combined with fluorescence curve melting analysis, Chmielewicz et al. rapidly classified the amplified virus to one of the six species. However, as with the PCR assays of Xu et al., Gu et al., and Wong et al., Chmielewicz et al.'s primers and probe combinations were not designed to discriminate between the different HAdV serotypes.

Real time PCR using primers from the hexon gene also exists for generic detection (but not discrimination) of all 51 serotypes (5, 12). Sequence analysis of a region in the hexon gene encoding the seven hypervariable loops (the primary antigenic determinant) can identify and discriminate all 51 serotypes (12, 16), however, the sequencing analysis is time consuming and requires the use of sequencing equipment and/or reagents.

Xu and Erdman designed a multiplex PCR assay for detecting and identifying HAdV-3, HAdV-7, and HAdV-21 (22). Xu and Erdman designed a pair of primers from the hypervariable region of the hexon gene for each of the HAdV-3, HAdV-7, and HAdV-21 serotypes. After subjecting the HAdV-3, HAdV-7, and HAdV-21 samples to a multiplex PCR reaction, the amplicons were distinguished by size on an agarose gel with ethidium bromide staining. However, the assay designed by Xu and Erdman is limited to detecting and identifying only three specific HAdV serotypes.

Timely serotype identification provides valuable epidemiological information and significantly contributes to treatment (antiviral) and prevention (vaccination) strategies. Unfortunately, conventional serotype identification is a tedious process, often taking days or longer. Although there are numerous assays available for detecting HAdV serotypes, and in some instances discriminating between up to three different HAdV stereotypes, there is currently no assay available that can conveniently and rapidly detect and identify four or more of any HAdV serotypes of interest, including the clinically relevant HAdV-3, HAdV-4, HAdV-7, HAdV-14, and HAdV-21 and/or HAdV-1, HAdV-2, HAdV-5, and HAdV-6.

### SUMMARY

The present disclosure provides methods and kits for conveniently and rapidly detecting and identifying four or more HAdV serotypes. In one embodiment, the methods and kits are designed for determining whether a sample contains one or more of at least HAdV-3, HAdV-4, HAdV-7, HAdV-14 and HAdV-21. In another embodiment, the method and kits are designed for determining whether a sample contains one or more of at least HAdV-1, HAdV-2, HAdV-5, and HAdV-6. In yet another embodiment, the methods and kits are designed for determining whether a sample contains one or more of at least HAdV-1, HAdV-2, HAdV-3, HAdV-4, HAdV-5, HAdV-6, HAdV-7, HAdV-14 and HAdV-21. Other HAdV serotypes of interest can be detected and identified using the methods and kits disclosed in this application.

In a specific embodiment, this disclosure provides a method of determining whether a sample contains one or more of HAdV-3, HAdV-4, HAdV-7, HAdV-14, and HAdV-21, wherein the sample comprises nucleic acid, the method comprising:

a) amplifying the nucleic acid in the sample using a first pair of primers and a second pair of primers, wherein the first pair of primers are designed to amplify a first region of a human adenovirus hexon gene and the second pair of primers are designed to amplify a second region of the human adenovirus hexon gene, and wherein if at least one of HAdV-3, HAdV-4, HAdV-14, or HAdV-21 is present in the test sample, a first amplification product is produced and if HAdV-7 is present in the sample a second amplification product is produced;

b) incubating any of the first or second amplification reaction products produced during the amplification step under hybridizing conditions with a first oligonucleotide probe (optionally comprising a first tag sequence), a second oligonucleotide probe (optionally comprising a second tag sequence), a third oligonucleotide probe (optionally comprising a third tag sequence), a fourth oligonucleotide probe (optionally comprising a fourth tag sequence), and a fifth oligonucleotide probe (optionally comprising a fifth tag sequence), wherein the first oligonucleotide probe is specific for HAdV-3, the second oligonucleotide probe is specific for HAdV-4, the third oligonucleotide probe is specific for HAdV-7, the fourth oligonucleotide probe is specific for HAdV-14, and the fifth oligonucleotide probe is specific for HAdV-21; and

c) detecting the presence of the first and/or second amplification product(s),

wherein if the sample contains HAdV-3, the first oligonucleotide probe hybridizes with the first amplification product produced by the first set of primers, thereby indicating the presence of the HAdV-3 serotype in the sample;

wherein if the sample contains HAdV-4, the second oligonucleotide probe hybridizes with the first amplification product produced by the first set of primers, thereby indicating the presence of the HAdV-4 serotype in the sample;

wherein if the sample contains HAdV-7, the third oligonucleotide probe hybridizes with the second amplification product produced by the second set of primers, thereby indicating the presence of the HAdV-7 serotype in the sample;

wherein if the sample contains HAdV-14, the fourth oligonucleotide probe hybridizes with the first amplification product produced by the first set of primers, thereby indicating the presence of the HAdV-14 serotype in the sample; and

wherein if the sample contains HAdV-21, the fifth oligonucleotide probe hybridizes with the first amplification product produced by the first set of primers, thereby indicating the presence of the HAdV-21 serotype in the sample.

In one embodiment, the one or more amplification products are detected simultaneously.

When the oligonucleotide probes comprise tag sequences, the method further comprises after the incubating step:

a) elongating any oligonucleotide probe hybridized to the first or second amplification product in the presence of a polymerase and four deoxyribonucleotide triphosphates to form an elongation product;

b) separating the elongation product from the first or second amplification product under denaturing conditions;

c) incubating the elongation product with a solid support under hybridizing conditions, wherein the solid support comprises a first capture oligonucleotide having a recognition sequence that is complementary to the first tag sequence in the first oligonucleotide probe, a second capture oligonucleotide having a recognition sequence that is complementary to the second tag sequence in the second oligonucleotide probe, a third capture oligonucleotide having a recognition sequence that is complementary to the third tag sequence in the third oligonucleotide probe, a fourth capture oligonucleotide having a recognition sequence that is complementary to the fourth tag sequence in the fourth oligonucleotide probe, and a fifth capture oligonucleotide having a recognition sequence that is complementary to the fifth tag sequence in the fifth oligonucleotide probe.

Alternatively, rather than comprising tag sequences, each of the first, second, third, fourth, and fifth oligonucleotide probe comprises a different label. Thus, in one embodiment, each oligonucleotide probe comprises a different fluorescent dye, making it possible to detect and discriminate between each of the HAdVs of interest in a single reaction.

In one embodiment, the detection step comprises analyzing the solid support to determine if the sample contains one or more of the HAdV-3 serotype, the HAdV-4 serotype, the HAdV-7 serotype, the HAdV-14 serotype, or the HAdV-21 serotype.

In another embodiment, the solid support comprises an array of microspheres, wherein the array of microspheres comprises a first microsphere comprising the first capture oligonucleotide, a second microsphere comprising the second capture oligonucleotide, a third microsphere comprising the third capture oligonucleotide, a fourth microsphere comprising the fourth capture oligonucleotide, and a fifth microsphere comprising the fifth capture oligonucleotide, and wherein each of the first, second, third, fourth, and fifth microspheres comprises a different fluorochrome or fluorescent dye.

In another embodiment, at least one of the deoxyribonucleotide triphosphates in the elongation step comprises a label, such as biotin. If biotin is used to label the elongation products, the method further comprises after incubating the elongation product with the solid support, adding avidin or streptavidin, wherein the avidin or streptavidin comprises a second label, such as a fluorescent dye.

In the detection step, the array of microspheres may be analyzed by flow cytometry to determine if the sample contains one or more of the HAdV-3 serotype, the HAdV-4 serotype, the HAdV-7 serotype, the HAdV-14 serotype, or the HAdV-21 serotype.

The methods described above could also be used to determine whether a sample contains one or more of HAdV-1, HAdV-2, HAdV-5, and HAdV-6 by using oligonucleotide probes specific for HAdV-1, HAdV-2, HAdV-5, and HAdV-6, such as those described herein. These serotypes are most commonly associated with adenovirus infections in pediatric and immunocompromised patients. These probes can be used in conjunction with the probes for HAdV-3, HAdV-4, HAdV-7, HAdV-14, and HAdV-21 to identify simultaneously the presence of one or more of HAdV-1, -2, -3, -4, -5, -6, -7, -14, and -21 in a single reaction. Alternatively, one could use the oligonucleotide probes specific for HAdV-1, HAdV-2, HAdV-5, and HAdV-6 in the methods described herein, using only the first pair of primers described above, to identify simultaneously one or more of HAdV-1, HAdV-2, HAdV-5, or HAdV-6 in a sample.

Another aspect is related to kits for identifying one or more of at least four or at least five HAdV serotypes in a sample. In one embodiment the HAdV serotypes comprise HAdV-1, HAdV-2, HAdV-5, and HAdV-6. In another embodiment, the HAdV serotypes comprise HAdV-3, HAdV-4, HAdV-7, HAdV-14, and HAdV-21. In yet another embodiment, the HAdV serotypes comprise HAdV-1, HAdV-2, HAdV-3, HAdV-4, HAdV-5, HAdV-6, HAdV-7, HAdV-14 and HAdV-21.

In one embodiment, the kit for identifying one or more of HAdV-3, HAdV-4, HAdV-7, HAdV-14, and HAdV-21 comprises

a) a first and second pair of primers, wherein the first pair of primers are designed to amplify a first region of a human adenovirus hexon gene and the second pair of primers are designed to amplify a second region of the human adenovirus hexon gene, and

b) a first, second, third, fourth, and fifth oligonucleotide probe, wherein the first oligonucleotide probe is specific for HAdV-3, the second oligonucleotide probe is specific for HAdV-4, the third oligonucleotide probe is specific for HAdV-7, the fourth oligonucleotide probe is specific for HAdV-14, and the fifth oligonucleotide probe is specific for HAdV-21.

In one embodiment of the kit (and the methods described herein), the first region of the human adenovirus hexon gene corresponds to about nucleotides 1003 to 1604 of the hexon gene of HAdV-21 of GenBank accession no. AY008279 (SEQ ID NO:1) and the second region of the human adenovirus hexon gene corresponds to about nucleotides 383 to 614 of the hexon gene of HAdV-21 of GenBank accession no. AY008279 (SEQ ID NO:1).

In another embodiment, the nucleotide sequences of the first pair of primers are CTGATGTACTACAACAGCACTGGCAACATGGG (SEQ ID NO:2) and CGGTGGTGGTTAAATGGATTCACATTGTCC (SEQ ID NO:3), and the nucleotide sequences of the second pair of primers are CGCCCAATACATCTCAGTGG (SEQ ID NO:4) and ACTCCAACTTGAGGCTCTGG (SEQ ID NO:5).

In another embodiment, each of the first, second, third, fourth, and fifth oligonucleotide probes has about 20-25 nucleotides, a G/C content of at least about 36%, and a melting temperature (Tᵢₙ) between about 50° C. and 56° C.

In another embodiment, the first oligonucleotide probe hybridizes under stringent conditions to the complement of a region of the HAdV-3 hexon gene corresponding to nucleotides 2,616 to 2,638 of the hexon gene of HAdV-3 of GenBank accession no. AY599834 (version AY599834.1 GI:57115749), the second oligonucleotide probe hybridizes under stringent conditions to the complement of a region of the HAdV-4 hexon gene corresponding to nucleotides 19,382 to 19,405 of the hexon gene of HAdV-4 of GenBank accession no. AY599837 (version AY599837.1 GI:57115887), the third oligonucleotide probe hybridizes under stringent conditions to the complement of a region of the HAdV-7 hexon gene corresponding to nucleotides 399 to 421 of the hexon gene of HAdV-7 of GenBank accession no. AY594255 (version AY594255.1 GI:51173294), the fourth oligonucleotide probe hybridizes under stringent conditions to the complement of a region of the HAdV-14 hexon gene corresponding to nucleotides 19,541 to 19,565 of the hexon gene of HAdV-14 of GenBank accession no. AY803294 (version AY803294.1 GI:57115621), and the fifth oligonucleotide probe hybridizes under stringent conditions to the complement of a region of the HAdV-21 hexon gene corresponding to nucleotides 1,299 to 1,318 of the hexon gene of HAdV-21 of GenBank accession no. AY008279 (version AY008279.1 GI:13919592).

In yet another embodiment, the nucleotide sequence of the first oligonucleotide probe, which is specific for HAdV-3, is GTTAAAACCGATGACACTAATGG (SEQ ID NO:6), the nucleotide sequence of the second oligonucleotide probe, which is specific for HAdV-4, is GGTGTGGGATTGACAGACACTTAC (SEQ ID NO:7), the nucleotide sequence of the third oligonucleotide probe, which is specific for HAdV-7, is GTGGATAGTTACAACGGGAGAAG (SEQ ID NO:8), the nucleotide sequence of the fourth oligonucleotide probe, which is specific for HAdV-14, is AGACCAAGCTTGGAAAGATGTAAAT (SEQ ID NO:9), and the nucleotide sequence of the fifth oligonucleotide, which is specific for HAdV-21, is GGGTGCAGATTGGAAAGAGC (SEQ ID NO: 10).

In another aspect, the kit for identifying one or more of HAdV-1, HAdV-2, HAdV-5, and HAdV-6 comprises

a) a pair of primers, wherein the pair of primers are designed to amplify a region of a human adenovirus hexon gene corresponding to about nucleotides 1003 to 1604 of the hexon gene of HAdV-21 of GenBank accession no. AY008279 (SEQ ID NO:1), and

b) a first, second, third, and fourth oligonucleotide probe, wherein the first oligonucleotide probe is specific for HAdV-1, the second oligonucleotide probe is specific for HAdV-2, the third oligonucleotide probe is specific for HAdV-5, and the fourth oligonucleotide probe is specific for HAdV-6.

In one embodiment, the nucleotide sequences of the first pair of primers are CTGATGTACTACAACAGCACTGGCAACATGGG (SEQ ID NO:2) and CGGTGGTGGTTAAATGGATTCACATTGTCC (SEQ ID NO:3).

In another embodiment, each of the first, second, third, fourth, and fifth oligonucleotide probes has about 20-25 nucleotides, a G/C content of at least about 40%, and a melting temperature (TO between about 50° C. and 56° C.

In another embodiment, the first oligonucleotide probe hybridizes under stringent conditions to the complement of a region of the HAdV-1 hexon gene corresponding to nucleotides 20,188 to 20,208 of the hexon gene of HAdV-1 having GenBank accession no. AF534906 (version AF534906.1 GI:33330439), the second oligonucleotide probe hybridizes under stringent conditions to the complement of a region of the HAdV-2 hexon gene corresponding to nucleotides 8,369 to 8,388 of the hexon gene of HAdV-2 having GenBank accession no. AC_{—}000007 (version AC_{—}000007.1 GI:56160492), the third oligonucleotide probe hybridizes under stringent conditions to the complement of a region of the HAdV-5 hexon gene corresponding to nucleotides 20,123 to 20,147 of the hexon gene of HAdV-5 having GenBank accession no. BK000408 (version BK000408.1 GI:33694637), and the fourth oligonucleotide probe hybridizes under stringent conditions to the complement of a region of the HAdV-6 hexon gene corresponding to nucleotides 1,321 to 1,339 of the hexon gene of HAdV-6 having GenBank accession no. AB330087 (version AB330087.1 GI190356534).

In yet another embodiment, the nucleotide sequence of the first oligonucleotide probe, which is specific for HAdV-1, is CAAACGGAAACGGTAATCCTC (SEQ ID NO:11), the nucleotide sequence of the second oligonucleotide probe, which is specific for HAdV-2, is CTAATGGCAATGGCTCAGGC (SEQ ID NO:12), the nucleotide sequence of the third oligonucleotide probe, which is specific for HAdV-5, is AAGGTAAAACCTAAAACAGGTCAGG (SEQ ID NO:13), and the nucleotide sequence of the fourth oligonucleotide probe, which is specific for HAdV-6, is GCTGCTAACGGGGACCAAG (SEQ ID NO:14).

The primers and probes of these kits can also be used in the methods described in this application.

Another aspect relates to an isolated oligonucleotide. In one embodiment, the isolated oligonucleotide is about 20 to 25 nucleotides in length and has a nucleotide sequence selected from:

(SEQ ID NO: 6)
(a) GTTAAAACCGATGACACTAATGG,
(SEQ ID NO: 7)
(b) GGTGTGGGATTGACAGACACTTAC,
(SEQ ID NO: 8)
(c) GTGGATAGTTACAACGGGAGAAG,
(SEQ ID NO: 9)
(d) AGACCAAGCTTGGAAAGATGTAAAT,
(SEQ ID NO: 10)
(e) GGGTGCAGATTGGAAAGAGC,
(SEQ ID NO: 11)
(f) CAAACGGAAACGGTAATCCTC,
(SEQ ID NO: 12)
(g) CTAATGGCAATGGCTCAGGC,
(SEQ ID NO: 13)
(h) AAGGTAAAACCTAAAACAGGTCAGG,
(SEQ ID NO: 14)
(i) GCTGCTAACGGGGACCAAG,
(SEQ ID NO: 15)
(j) AACAAGCGAGTGGTGGCTC
(SEQ ID NO: 4)
(k) CGCCCAATACATCTCAGTGG,
(SEQ ID NO: 5)
(l) ACTCCAACTTGAGGCTCTGG,
or
(m) the complement of any one of (a), (b), (c),
(d), (e), (f), (g), (h), (i), or (j).

The isolated oligonucleotide optionally comprises a label.

### DETAILED DESCRIPTION

Reference will now be made in detail to various exemplary embodiments. It is to be understood that the following detailed description is provided to give the reader a fuller understanding of certain embodiments, features, and details of aspects of the invention, and should not be interpreted as a limitation of the scope of the invention.

### 1. DEFINITIONS

In order that the present invention may be more readily understood, certain terms are first defined. Additional definitions are set forth throughout the detailed description.

As used herein, “nucleic acid,” “nucleotide sequence,” or “nucleic acid sequence” refer to a nucleotide, oligonucleotide, polynucleotide, or any fragment thereof and to naturally occurring or synthetic molecules. These phrases also refer to DNA or RNA of genomic or synthetic origin which may be single-stranded or double-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all occurrences of the nitrogenous base thymine are replaced with uracil, and the sugar backbone is composed of ribose instead of deoxyribose. RNA may be used in the methods described herein and/or may be converted to cDNA by reverse-transcription for use in the methods described herein. Methods for reverse transcription are well known in the art. See, e.g., See Maniatis et al., Molecular Cloning, A Laboratory Manual, 2d, Cold Spring Harbor Laboratory Press, pages 16-54 (1989).

As used herein, the term “oligonucleotide” refers to a short polymer composed of deoxyribonucleotides, ribonucleotides or any combination thereof. Oligonucleotides are generally between about 10, 11, 12, 13, 14 or 15 to about 150 nucleotides (nt) in length, preferably about 10, 11, 12, 13, 14, or 15 to about 70 nt, and more preferably between about 18 to about 30 nt in length.

The term “capture oligonucleotide” refers to an oligonucleotide having a nucleic acid recognition sequence and coupled to a solid surface to hybridize with an oligonucleotide probe having a nucleic acid “tag sequence” complementary to the recognition sequence, thereby capturing the oligonucleotide probe on the solid surface. Each serotype specific oligonucleotide probe has its own unique tag sequence.

The term “complement,” “complementary,” or “complementarity” as used herein with reference to a nucleic acid sequence refers to standard Watson Crick pairing rules. The complement of a nucleic acid sequence such that the 5′ end of one sequence is paired with the 3′ end of the other, is in antiparallel association. For example, the sequence 5′-A-G-T-3′ is complementary to the sequence 3′-T-C-A-5′. Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “complementary” as used herein means that two sequences specifically hybridize (defined below). The skilled artisan will understand that complementary sequences need not hybridize along their entire length.

“Specific hybridization,” “specifically hybridize,” or “specific for” is an indication that two nucleic acid sequences share a high degree of complementarity. An oligonucleotide (e.g., a probe or a primer) that is specific for a target nucleic acid will hybridize to the target nucleic acid (e.g., the hexon gene of a specific HAdV serotype of interest) under suitable conditions and will not substantially hybridize to nucleic acids that are not of interest (e.g., the hexon gene of a HAdV serotype other than the HAdV serotype of interest) under those same conditions. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and amplification. The term “primer” as used herein includes all forms of primers that may be synthesized including peptide nucleic acid primers, locked nucleic acid primers, phosphorothioate modified primers, labeled primers, and the like. As used herein, a “forward primer” is a primer that is complementary to the anti-sense strand of dsDNA. A “reverse primer” is complementary to the sense-strand of dsDNA. Primers are typically between about 10 and about 100 nucleotides in length, preferably between about 12 and about 30 nucleotides in length, and most preferably between about 15 and about 25 nucleotides in length. There is no standard length for optimal hybridization or polymerase chain reaction amplification. An optimal length for a particular primer application may be readily determined by the skilled artisan, such as in the manner described in H. Erlich, PCR Technology, Principles and Application for DNA Amplification (1989).

The term “isolated,” when used in the context of a nucleic acid sequence, refers to a nucleic acid that is substantially free from a component that normally accompanies the nucleic acid in its natural environment. For instance, an isolated nucleic acid is substantially free of cellular material and/or other nucleic acids from the cell or tissue source from which it was derived. A skilled artisan can readily employ nucleic acid isolation procedures to obtain an isolated nucleic acid.

### 2. AMPLIFICATION OF NUCLEIC ACIDS

In one aspect of the methods described herein, a HAdV nucleic acid is amplified from a biological sample containing nucleic acids. Nucleic acid samples or isolated nucleic acids may be amplified by various methods known to the skilled artisan. The nucleic acid (DNA or RNA) may be isolated from the sample according to any methods well known to those of skill in the art. If necessary the sample may be collected or concentrated by centrifugation and the like. If the sample contains cells, the cells of the sample may be subjected to lysis, such as by treatments with enzymes, heat surfactants, ultrasonication or combination thereof. The lysis treatment is performed in order to obtain a sufficient amount of DNA from the sample to detect using a nucleic acid amplification reaction.

Various methods of DNA extraction are suitable for isolating the DNA. Suitable methods include phenol and chloroform extraction. See Maniatis et al., Molecular Cloning, A Laboratory Manual, 2d, Cold Spring Harbor Laboratory Press, page 16-54 (1989). Numerous commercial kits also yield suitable DNA including, but not limited to, QIAamp™ mini blood kit, Agencourt Genfind™, Roche Cobas® or phenol:chloroform extraction using Eppendorf Phase Lock Gels®.

Preferably, PCR is used to amplify nucleic acids of interest. Briefly, in PCR, two primer sequences are prepared that are complementary to regions on opposite complementary strands of the target sequence. An excess of deoxynucleotide triphosphates (dNTPs) are added to a reaction mixture along with a DNA polymerase, e.g. Taq polymerase. If the target nucleic acid is present in a sample, the primers will bind to the sequence and the polymerase will cause the primers to be extended along the target sequence by adding on nucleotides. By raising and lowering the temperature of the reaction mixture, the extended primers will dissociate from the marker to form reaction products, excess primers will bind to the marker and to the reaction products and the process is repeated, thereby generating amplification products. Cycling parameters can be varied, depending on the length of the amplification products to be extended.

### 3. NUCLEIC ACIDS

The present disclosure further provides isolated nucleic acids corresponding to the primers and oligonucleotide probes that can be used in the methods described herein. The nucleic acids may comprise DNA or RNA and may be wholly or partially synthetic or recombinant. Reference to a nucleotide sequence as set out herein encompasses a DNA molecule with the specified sequence, and encompasses a RNA molecule with the specified sequence in which U is substituted for T, unless context requires otherwise.

### 4. SOLID SUPPORTS

In certain embodiments, methods are carried out, at least in part, using a solid support. A variety of different supports can be used. In some embodiments, the solid support is a single solid support, such as a chip or wafer, or the interior or exterior surface of a tube, cone, or other article. In some embodiments, oligonucleotide probes or capture oligonucleotides may be immobilized at defined positions on the solid support to generate a two dimensional array. The solid support is fabricated from any suitable material to provide an optimal combination of such desired properties as stability, dimensions, shape, and surface smoothness. Preferred materials do not interfere with nucleic acid hybridization and are not subject to high amounts of non-specific binding of nucleic acids. Suitable materials include biological or nonbiological, organic or inorganic materials. For example, an array can be fabricated from any suitable plastic or polymer, silicon, glass, ceramic, or metal, and can be provided in the form of a solid, resin, gel, rigid film, or flexible membrane. Suitable polymers include, for example, polystyrene, poly(alkyl)methacrylate, poly(vinylbenzophenone), polycarbonate, polyethylene, polypropylene, polyamide, polyvinylidenefluoride, and the like. Preferred materials include polystyrene, glass, and silica. In a particular embodiment, the solid support is a film-based two-dimensional microarray such as the BioFilmChip™ available from AutoGenomics (Carlsbad, Calif.).

Dimensions of the solid support are determined based upon such factors as the desired number of regions and the number of sequences to be assayed. As an example, a solid support can be provided with planar dimensions of about 0.5 cm to about 7.5 cm in length, and about 0.5 cm to about 7.5 cm in width. Solid supports can also be singly or multiply positioned on other supports, such as microscope slides (e.g., having dimensions of about 7.5 cm by about 2.5 cm). The dimensions of the solid support can be readily adapted for a particular application.

In some embodiments, the solid support is a particulate support, also referred to as a microsphere, bead or particle. In particular embodiments, the particles are conjugated directly to the oligonucleotide probes or the capture oligonucleotides. Typically, the particles form groups in which particles within each group have a particular characteristic, such as, for example, color, fluorescence frequency, density, size, or shape, which can be used to distinguish or separate those particles from particles of other groups. Preferably, the particles can be separated using techniques, such as, for example, flow cytometry.

The particles can be fabricated from virtually any insoluble or solid material. For example, the particles can be fabricated from silica gel, glass, nylon, resins, Sephadex™ Sepharose™, cellulose, magnetic material, a metal (e.g., steel, gold, silver, aluminum, copper, or an alloy) or metal-coated material, a plastic material (e.g., polyethylene, polypropylene, polyamide, polyester, polyvinylidenefluoride (PVDF)) and the like, and combinations thereof. Examples of suitable micro-beads are described, for example, in U.S. Pat. Nos. 5,736,330, 6,046,807, and 6,057,107, all of which are incorporated herein by reference in their entirety. Examples of suitable particles are available, for example, from Luminex Corp., Austin, Tex.

In certain embodiments, the support (whether a two-dimensional array or particulate support) is capable of binding or otherwise holding an oligonucleotide probe or a capture oligonucleotide to the surface of the support in a sufficiently stable manner to accomplish the purposes described herein. Such binding can include, for example, the formation of covalent, ionic, coordinative, hydrogen, or van der Waals bonds between the support and the oligonucleotide probe or the capture oligonucleotide or attraction to a positively or negatively charged support. Oligonucleotide probes or capture oligonucleotides are attached to the solid support surface directly or via linkers. In one embodiment, oligonucleotide probes or capture oligonucleotides are directly attached to the support surface by providing or derivatizing either the surface, the oligonucleotide probe/capture oligonucleotide, or both, with one or more reactive groups. In one embodiment, well-known chemical crosslinkers may be used for covalent linkage. For example, amino-labeled primers can be covalently attached to carboxylated solid supports using N-(3-Dimethylaminopropyl)-N′-ethylcarbodiimide hydrochloride (EDAC). In another example, the surface of Luminex™ particles can be modified with, for example, carboxylate, maleimide, or hydrazide functionalities or avidin and glass surfaces can be treated with, for example, silane or aldehyde (to form Schiff base aldehyde-amine couplings with DNA). In some embodiments, the support or a material disposed on the support (as, for example, a coating on the support) includes reactive functional groups that can couple with a reactive functional group on the oligonucleotide probe or the capture oligonucleotide. As examples, the support can be functionalized (e.g., a metal or polymer surface that is reactively functionalized) or contain functionalities (e.g., a polymer with pending functional groups) to provide sites for coupling the oligonucleotide probes or the capture oligonucleotides.

In other embodiments, the support can be partially or completely coated with a binding agent, such as streptavidin, antibody, antigen, enzyme, enzyme cofactor or inhibitor, hormone, or hormone receptor. The binding agent is typically a biological or synthetic molecule that has high affinity for another molecule or macromolecule, through covalent or non-covalent bonding. The oligonucleotide probe or the capture oligonucleotide is coupled to a complement of the binding agent (e.g., biotin, antigen, antibody, enzyme cofactor or inhibitor, enzyme, hormone receptor, or hormone). The oligonucleotide probe or the capture oligonucleotide is then brought in contact with the binding agent to hold the oligonucleotide probe or the capture oligonucleotide on the support. Other known coupling techniques can be readily adapted and used in the systems and methods described herein.

In one embodiment, the solid support comprises microspheres uniquely distinguished by detectable characteristics. The microspheres are alternately termed microparticles, beads, polystyrene beads, microbeads, latex particles, latex beads, fluorescent beads, fluorescent particles, colored particles and colored beads. The microspheres serve as vehicles for molecular reactions. Microspheres for use in flow cytometry may be obtained from manufacturers, such as Luminex Corp. of Austin, Tex. Illustrative microspheres and methods of manufacturing same are, for example, found in U.S. Pat. Nos. 6,268,222 and 6,632,526, which are incorporated herein by reference in their entirety.

Microspheres may be composed of polystyrene, cellulose, or other appropriate material. In a particular embodiment, microspheres are stained with different amounts of fluorescent dyes. Preferably the dyes have the same or overlapping excitation spectra, but possess distinguishable emission spectra. Fluorescent dyes that may be used in the microspheres include cyanine dyes, with emission wavelengths between 550 nm and 900 nm. These dyes may contain methine groups and their number influences the spectral properties of the dye. The monomethine dyes that are pyridines typically have blue to blue-green fluorescence emission, while quinolines have green to yellow-green fluorescence emission. The trimethine dye analogs are substantially shifted toward red wavelengths, and the pentamethine dyes are shifted even further, often exhibiting infrared fluorescence emission (see for example U.S. Pat. No. 5,760,201). However, any dye that is soluble in an organic solvent can be used.

The classification parameters of each microsphere advantageously include one, two, three, four, or more standard fluorochromes or fluorescent dyes. The one or more fluorochromes are affixed to or embedded in each microsphere by any standard method, for example, by attachment to the microsphere surface by covalent bonding or adsorption. Alternatively, the dye(s) may be affixed by a copolymerization process, wherein monomers, such as an unsaturated aldehyde or acrylate, are allowed to polymerize in the presence of a fluorescent dye, such as fluorescein isothiocynate (FITC), in the resulting reaction mixture.

Another method by which one or more dyes are embedded in a microsphere includes adding a subset of microspheres to, for example, an organic solvent to expand the microspheres. An oil-soluble or hydrophobic dye, for example, is subsequently added to the subset of microspheres, thereby penetrating into each microsphere. After incubating the resulting combination, an alcohol or water-based solution, for example, is added to the combination and the organic solvent is removed. The microsphere shrinks, retaining the dye(s) inside. Each fluorochrome in the microsphere optionally serves as an additional or alternative classification parameter.

Each of the microspheres is addressed to a unique capture oligonucleotide, permitting the analysis of many different probes in a single reaction. After a single reaction, the particles are supplied to a reader system, which determines the particle IDs to identify the particle types and also to detect the reporter signals. The reader system includes multiple excitation light sources, such as laser or other devices with controlled wavelengths and optical power, such as LEDs, SLDs, broadband sources with excitation filters, and so forth. The light sources excite the various reporters to supply associated signals to one or more detectors. Emission filters and wavelength discriminators are included such that a given detector receives at a given time the signals associated with a single assay binding label.

### 5. CAPTURE AND TAG OLIGONUCLEOTIDES

In one embodiment, each oligonucleotide probe has a unique tag nucleic acid sequence, which is complementary to a unique nucleic acid recognition sequence in a capture oligonucleotide conjugated to the solid support (e.g., microsphere or two-dimensional array). Thus, the capture oligonucleotide includes a recognition sequence that can capture, by hybridization, an oligonucleotide probe having a complementary tag sequence. The hybridization of the recognition sequence of a capture oligonucleotide and the tag sequence of an oligonucleotide probe results in the coupling of the oligonucleotide probe to the solid support. The recognition sequence of a particular capture oligonucleotide and its complementary tag sequence are, thus, associated with a single, specific HAdV serotype.

The recognition and tag sequences typically include at least six nucleotides and, in some instances, include at least 8, 10, 15, or 20 or more nucleotides. The capture oligonucleotide also typically includes a functional group that permits binding of the capture oligonucleotide to the solid support or functional groups disposed on or extending from the solid support. The functional group can be attached directly to the polymeric backbone or can be attached to a base in the nucleotide sequence. As an alternative, the capture oligonucleotide can include a crosslinking portion to facilitate crosslinking, as described above, or can be electrostatically held on the surface. The capture oligonucleotides can be formed by a variety of techniques, including, for example, solid state synthesis, DNA replication, reverse transcription, restriction digest, run-off transcription, and the like. Commercial capture and linker sequence sets are provided by TagIt™ (Luminex, Austin, Tex.) and ZipCode™ (Celera, Rockville, Md.)

In one embodiment, solid supports with associated capture oligonucleotides are disposed in a holder, such as, for example, a vial, tube, or well. After incubating the oligonucleotide probes with the amplification reaction products, followed by an elongation step, the elongated products are added to the holder under hybridization conditions. The elongation step optionally includes incorporating a detectable label into the elongation product, for example, by using a deoxyribonucleotide triphosphate comprising the detectable label. The groups of supports are then investigated to determine which support(s) have elongation products attached thereto. Optionally, the supports can be washed to reduce the effects of cross-hybridization. One or more washes can be performed at the same or different levels of stringency. As another optional alternative, prior to contact with the support(s) and capture oligonucleotides, the solution containing elongation products can be subjected to, for example, size exclusion chromatography, differential precipitation, spin columns, or filter columns to remove oligonucleotide probes that have not been elongated or to remove other materials that are not the same size as the elongation products.

### 6. TARGET SPECIFIC PRIMER ELONGATION (TSPE)

In one embodiment, following the nucleic acid amplification reaction, the amplification products, or amplicons, are denatured and contacted under hybridization conditions with oligonucleotide probes that are specific for the HAdV serotypes of interest. The oligonucleotide probes may be bound to a solid support or free in solution. In the presence of a polymerase, the oligonucleotide probes function as primers, and the polymerase catalyzes the addition of nucleotides to the 3′ end of the oligonucleotide probes. Thus, a TSPE reaction includes a primer to be extended (i.e., a HAdV-specific oligonucleotide probe), a template (i.e., one of the strands of an amplification reaction product), a polymerase, and the four deoxyribonucleotide triphosphates (dATP, dGTP, dTTP, and dCTP). Preferably, at least one of the deoxyribonucleotide triphosphates comprises a label. That way any elongation product generated from the TSPE reaction will include the label, facilitating the detection of the elongation products.

### 7. DETECTION OF HADV SEROTYPES BY FLOW CYTOMETRY

In one embodiment, flow cytometry is used to analyze the reaction product(s) of the amplification and/or elongation reaction. Flow cytometry is capable of sensitive and quantitative fluorescence measurements of individual particles without the need to separate free from particle-bound label. Analysis rates are very high (hundreds to thousands of particles per second), and multiple fluorescence and light scatter signals can be detected simultaneously.

In some embodiments, the methods use encoded particles or microspheres, having a particular detectable signature, that are conjugated to a unique capture oligonucleotide to form particle types. The sets of particle types are then pooled, and aliquots of the particle types are removed to assay vessels. Samples of labeled reaction products from the elongation reaction are supplied to the respective vessels. Following hybridization, the encoded particles and labeled reaction products can be detected using a flow cytometer that is capable of determining both the identity of the microsphere and of the labeled reaction product. A computer may be used to associate the signals generated from the particle ID signature and the labeled reaction product with a specific HAdV serotype.

For each microsphere product supplied, the reader system determines the particle ID and the presence or absence or the labeled reaction product. Each particle ID is associated with a capture oligonucleotide that includes a unique recognition sequence that can capture, by hybridization, an oligonucleotide probe, or an elongation product derived there from, having a complementary tag sequence. Using this information, a sample can be assayed to determine whether it contains HAdV and, if so, to determine simultaneously the identity of the one or more HAdV serotypes present in the sample.

### 8. DETECTION OF HADV SEROTYPES USING TWO-DIMENSIONAL ARRAY SCANNING

In one embodiment of the invention, the solid support is a two-dimensional microarray or biochip. The oligonucleotide probes or unique capture oligonucleotides are immobilized on the array at predetermined positions. In some embodiments, the oligonucleotide probe may be elongated or extended on the array. In other embodiments, the oligonucleotide probes are elongated or extended before, after, or during hybridization to the array. Each address on the array is associated with a capture oligonucleotide or oligonucleotide probe specific for a single HAdV serotype. Using this information, the identity of the one or more HAdV serotypes in a sample can be determined simultaneously.

In one embodiment, the microarray of the present invention comprises a film-based microarray such as the BioFilmChip™ available from AutoGenomics (Carlsbad, Calif.). These biochips comprise a matrix layer coupled to a substrate, wherein the matrix layer includes a plurality of oligonucleotides in a plurality of predetermined positions. The term “predetermined position” of an analyte refers to a particular position of the analyte on the chip that is addressable by at least two coordinates relative to a registration marker on the chip, and particularly excludes a substantially complete coating of the chip with the analyte and/or probe. Therefore, preferred pluralities of predetermined positions will include an array with a multiple rows of substrates forming multiple columns.

In some embodiments, matrix layers may be multi-functional matrix layers that reduce autofluorescence, incident-light-absorption, charge-effects, and/or surface unevenness of the substrate, and contemplated biochips may comprise additional matrix layers. This microarray may be used with a platform such as the Infiniti™ Analyzer, also available from AutoGenomics (Carlsbad, Calif.). Other suitable approaches include the microarray technology commercially available from a variety of sources, such as microarray products available from Affymetrix (Santa Clara, Calif.), including, for example, the Affymetrix GeneChip® arrays.

### 9. SINGLEPLEX AND MULTIPLEX ASSAYS

At least one oligonucleotide probe is provided for each HAdV serotype of interest. In one aspect only one oligonucleotide probe is provided for each HAdV serotype of interest. In some embodiments, the oligonucleotide probes are incubated with the amplification reaction products in a singleplex format, i.e., each oligonucleotide probe is provided in a separate reaction vessel. In other embodiments, the oligonucleotide probes are incubated with the amplification reaction products in a multiplex format, i.e., in the same reaction vessel. Variations of these embodiments include reactions where some oligonucleotide probes of interest are provided in one reaction vessel and other oligonucleotide probes of interest are provided in separate reaction vessels. Similarly, in one aspect, the methods described herein are designed for simultaneous amplification of multiple targets, using two pairs of primers, in a multiplex format i.e., in the same reaction vessel.

### 10. LABEL

The label used in the methods and compositions described herein can be any detectable moiety, such as a fluorescent compound. Exemplary fluorescent compounds include 4-acetamido-4′-isothiocyanatostilbene-2,2′ disulfonic acid, acridine and derivatives (acridine, acridine isothiocyanate) Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes), 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow V5), N-(4-anilino-1-naphthyl)maleimide, anthranilamide, Black Hole Quencher™ (BHQ™) dyes (biosearch Technologies), BODIPY® R-6G, BOPIPY® 530/550, BODIPY® FL, Brilliant Yellow, coumarin and derivatives (coumarin, 7-amino-4-methylcoumarin (AMC, Coumarin 120), 7-amino-4-trifluoromethylcouluarin (Coumarin 151)), Cy2®, Cy3®, Cy3.5®, Cy5®, Cy5.5®, cyanosine, 4′,6-diaminidino-2-phenylindole (DAPI), 5′,5″-dibromopyrogallol-sulfonephthalcin (Bromopyrogallol Red), 7-diethylamino-3-(4′-isothiocyanatophenyl)-4-methylcoumarin, diethylenetriamine pentaacetate, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), 4-(4′-dimethylaminophenylazo)benzoic acid (DABCYL), 4-dimethylaminophenylazophenyl-4′-isothiocyanate (DABITC), Eclipse™ (Epoch Biosciences Inc.), eosin and derivatives (eosin, eosin isothiocyanate), erythrosin and derivatives (erythrosin B, erythrosin isothiocyanate), ethidium, fluorescein and derivatives (5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET)), fluorescamine, IR144, IR1446, Malachite Green isothiocyanate, 4-methylumbelliferone, ortho cresolphthalein, nitrotyrosine, pararosaniline, Phenol Red, B-phycoerythrin, R-phycoerythrin, o-phthaldialdehyde, Oregon Green®, propidium iodide, pyrene and derivatives (pyrene, pyrene butyrate, succinimidyl 1-pyrene butyrate), QSY® 7, QSY® 9, QSY® 21, QSY® 35 (Molecular Probes), Reactive Red 4 (Cibacron® Brilliant Red 3B-A), rhodamine and derivatives (6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, sulforhodamine D, sulforhodamine 101, sulfonyl chloride derivative of sulforhodamine 101 (Texas Red)), N,N,N′,N′-tetramethyl-6-carboxyrhodamine (TAMRA), tetramethyl rhodamine, tetramethyl rhodamine isothiocyanate (TRITC), riboflavin, rosolic acid, terbium chelate derivatives.

The detectable moiety may also be a bioluminescent compound (such as luciferase, green fluorescent protein (GFP), yellow fluorescent protein, etc.); an enzyme that produces a detectable reaction product (such as horseradish peroxidase, β-galactosidase, luciferase, alkaline phosphatase, or glucose oxidase and the like); or a radiolabel (such as ³H, ¹⁴C, ¹⁵N, ³⁵S, ⁹⁰Y, ⁹⁹Tc, ¹¹¹In, ¹²⁵I, ¹³¹I).

### 11. KITS

The HAdV oligonucleotide probes and/or primers as described herein may be supplied in the form of a kit useful, for example, for performing the methods described in this application. For example, a kit can comprise in packaged combination with other reagents any or all of the primers or oligonucleotide probes described herein. Generally, it is desirable to include the requisite number of probes and/or primers to afford identification of all of the HAdV serotypes of interest. The oligonucleotide probes can be packaged to permit the assay to be performed in a hetero- or homogeneous format. The oligonucleotide probes can be labeled or bound to a support or can be provided with groups that permit the probe or primer to be subsequently labeled or bound to a support.

In one embodiment, the kit includes two pairs of primers for amplifying different regions of a HAdV hexon gene and/or a single oligonucleotide probe for each HAdV serotype of interest. Thus, in one embodiment the kit includes two pairs of primers for amplifying different regions of a HAdV hexon gene and/or five oligonucleotide probes, one specific for each of HAdV-3, HAdV-4, HAdV-7, HAdV-14, and HAdV-21. In another embodiment, the kit includes a pair of primers for a region of a HAdV hexon gene and/or four oligonucleotide probes, one specific for each of HAdV-1, HAdV-2, HAdV-5, and HAdV-6. In yet another embodiment, the kit includes two pairs of primers for amplifying different regions of a HAdV hexon gene and/or nine oligonucleotide probes, one specific for each of HAdV-1, HAdV-2, HAdV-3, HAdV-4, HAdV-5, HAdV-6, HAdV-7, HAdV-14, and HAdV-21.

In other embodiments, a solid support, such as a two-dimensional array or microsphere, may be provided in an appropriate and separate container. Other kit embodiments include a buffer and/or components (e.g., a polymerase, dNTPs, etc.) for conducting a nucleic acid amplification reaction.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Example 1

### Rapid and Sensitive HAdV Multiplex Assay for Detecting and Identifying Serotypes 3, 4, 7, 14, and 21

Sample collection and initial identification. One-hundred five clinical specimens previously identified as HAdV serotypes, 3, 4, 7, 11, 14, and 21 were provided by the Naval Health Research Center under an IRB protocol (NHRC.1999.0002) with support from the Armed Forces Health Surveillance Center (AFHSC)/Division of Global Emerging Infections Surveillance and Response System (GEIS) under work unit 60805. Inclusion criteria for consented subjects enrolled through the NHRC febrile respiratory illness surveillance system included military recruits reporting for medical care with respiratory symptoms and a fever of =38° C. Samples included oropharyngeal swabs suspended in Viral Transport Medium (VTM), (Copan Diagnostics Inc., Murrieta, Calif.) and subsequently frozen at −80° C. and transported on dry ice for testing. The presence of HAdVs serotypes 3, 4, 7, 14 and 21 was detected in 10, 19, 10, 19 and 20 respectively by PCR and/or samples were initially identified at NHRC using a microneutralization assay, PCR, or both methods as described previously (10). These samples were collected, stored, and transported under College of American Pathologists (CAP) accredited diagnostic protocols.

Viral Strains and Isolates.

HAdV isolates used in this study were part of the strain bank from the Division of Viral Disease at WRAIR, and were grown using A549 cells as previously described (26, 27). The following HAdV strains were used: HAdV-1 (Adenoid 71), HAdV-2 (Adenoid 6), HAdV-3 (GB), HAdV-4 (RI-67), HAdV-5 (Adenoid 75), HAdV-6 (Tonsil 99), HAdV-7a (S-1058), HAdV-7 (Gomen), HAdV9 (Hick), HAdV-11 (Slobitski), HAdV-14 (DeWit), HAdV-16 (CH76), HAdV-17, HAdV-21 (128), HAdV-31 (1315/63), HAdV-34 (Compton), HAdV-35 (Holden), and HAdV-40 (Dugan). Cultures from strains GB, RI-67, S-1058, Gomen, DeWit, and 128 HAdV were titrated in tube cultures for 21 days and titers were expressed in 50% tissue culture infective dose (TCID₅₀).

Other common respiratory pathogens were obtained using the Zeptometrix, Respiratory Validation Panel NATrol™ (Zeptometrix, Buffalo, N.Y.). This panel includes Corona OC43, Corona SARS, Influenza A H1N1 and H3N2, Influenza B, Parainfluenza 2 and 3, Adenovirus 7a Metapneumovirus, Respiratory Syncytial Virus (RSV) A and B, Enterovirus and Rhinovirus.

DNA Extraction.

Oropharyngeal swabs and cultured isolates were processed under BSL-2 conditions at the Division of Viral Disease at WRAIR. DNA was isolated using the MinElute® Virus Spin kit (Qiagen, Valencia, Calif.) according to the manufacturers' recommendations. The sample and elution volumes were 200 μl. Sample extracts were stored at −70° C. The presence and quality of the nucleic acids from each extract was confirmed using xTAG® Respiratory Viral Panel (Luminex, Austin, Tex.).

Primer and Probe Design.

With molecular multiplexed assays there is the concern of decreased sensitivity. Primers and probes for each target may interfere with one another by forming dimers and or partial binding to target sequences. This can be minimized by the primer and probe design and the sequence variability among the targets. This assay was designed to minimize the number of primers and probes needed to amplify and identify the targets of interest.

For PCR primer and oligonucleotide probe design, eight hexon gene sequences from the serotypes of interest (GenBank accession numbers AY599834, AY599836, AY599837, AY594255, AF065066, AY495969, AY803294 and AY008279) were aligned by DNAStar®, Lasergene 8.0 software. A primer pair previously described was used to amplify a 605 base pair region of the hexon gene (16). The nucleotide sequences of the primer pair are as follows:

HVR7′ Forward:
(SEQ ID NO: 2)
CTGATGTACTACAACAGCACTGGCAACATGGG;
and
HVR7′ Reverse:
(SEQ ID NO: 3)
CGGTGGTGGTTAAATGGATTCACATTGTCC.

The HVR7′ forward primer and HVR7′ reverse primer correspond to nucleotides 1003-1033 and 1575-1604, respectively, of the 2850 base pair, HAdV-21 hexon gene having the GenBank accession number AY008279 (version AY008279.1 GI:1391959′2), the sequence of which is:

| | (SEQ ID NO: 1) |
|---|---|
| 1 | atggccaccc catcgatgct gccccagtgg gcatacatgc acatcgccgg acaggatgct |
| 61 | tcggagtacc tgagtccggg tctggtgcag ttcgcccgcg ccacagacac ctacttcaat |
| 121 | ctggggaaca agtttaggaa ccccaccgtg gcgcccaccc atgatgtgac caccgaccgc |
| 181 | agtcagcggc tgatgctgcg ctttgtgccc gttgaccggg aagacaatac ctacgcatac |
| 241 | aaagttcgat acaccttggc tgtgggcgac aacagagtgc tggatatggc cagcactttc |
| 301 | tttgacattc ggggtgtgtt ggatagaggc cctagcttca agccatactc tggcactgct |
| 361 | tacaactcgt tggcccctaa gggcgctccc aatacatctc agtggattgc tgaaggcgta |
| 421 | aaaaaagaag atgggggatc tgacgaagag gaagagaaaa atctcaccac ttacactttt |
| 481 | ggaaatgccc cagtgaaagc agaaggtggt gatatcacta aagacaaagg tcttccaatt |
| 541 | ggttcagaaa ttacagacgg cgaagccaaa ccaatttatg cagataaact ataccaacca |
| 601 | gaacctcagg tgggagatga aacttggact gacacagatg gaacaactga gaagtatggt |
| 661 | ggtagagctc taaagccaga aactaaaatg aaaccctgct atgggtcttt tgctaaaccc |
| 721 | actaacgtca aaggcggaca ggcaaaacaa aaaactactg aacaaccgca aaaccagcag |
| 781 | gttgaatatg atattgacat gaactttttt gatgaagcgt cacagaaagc aaacttcagt |
| 841 | ccaaaaattg tgatgtatgc agaaaatgta gacttggaaa ccccagacac tcatgtggtg |
| 901 | tacaaacctg gtacttcaga agaaagttct catgctaatc tgggtcaaca atctatgccc |
| 961 | aacagaccca actacattgg ctttagagat aactttattg gacttatgta ctacaacagt |
| 1021 | actggcaaca tgggagtgct ggcaggtcaa gcatcccaat tgaatgcggt ggttgacttg |
| 1081 | caggacagaa acacagaact atcatatcaa ctactgcttg actctctggg tgacagaacc |
| 1141 | agatacttca gcatgtggaa tcaagcagtc gatagctatg atcctgatgt gcgcattatt |
| 1201 | gaaaatcatg gggtggaaga tgagcttccc aactactgct ttccattgga tggagtaggg |
| 1261 | gtaccaataa gtagttacaa aataattgaa ccaaacggac agggtgcaga ttggaaagag |
| 1321 | cctgacataa atggaacaag tgaaattgga caaggaaatc tctttgccat ggaaattaac |
| 1381 | ctccaagcta atctctggag aagttttctt tattccaatg tggctctgta tctcccagac |
| 1441 | tcctacaaat acaccccagc caatgtcact cttccaacta acaccaacac ttatgactac |
| 1501 | atgaatgggc gggtggttcc cccatccctg gtggatacct acgtaaacat tggcgccaga |
| 1561 | tggtctttgg atgccatgga caatgtcaac ccctttaacc atcaccgcaa cgctggcctg |
| 1621 | cgataccggt ccatgctttt gggcaatggt cgttacgtgc ctttccacat tcaagtgcct |
| 1681 | cagaaattct ttgctgtgaa gaacctgctg cttctacccg gttcttacac ctacgagtgg |
| 1741 | aacttcagaa aggatgtgaa catggtcctg cagagttccc ttggtaatga tctccgggtc |
| 1801 | gatggtgcca gcataagttt taccagcatc aatctctatg ccaccttctt ccccatggcc |
| 1861 | cacaacactg cctccaccct tgaagccatg ctgcgcaatg acaccaatga tcaatcattc |
| 1921 | aatgactacc tttctgctgc caacatgctc taccccatcc cggccaacgc taccaacgtt |
| 1981 | cccatctcca ttccctctcg caactgggcc gccttcagag gctggtcctt caccagactc |
| 2041 | aaaaccaagg agactccctc tttgggatca gggttcgatc cctactttgt ttactctggt |
| 2101 | tctataccct acctggatgg taccttctac cttaaccaca ctttcaagaa agtctccatc |
| 2161 | atgtttgact cttcagtgag ctggcctggt aatgacagat tgctaagtcc aaatgagttc |
| 2221 | gaaatcaagc gcacagttga tggggaaggc tacaatgtgg cccaatgtaa catgaccaaa |
| 2281 | gactggttcc tggtccagat gcttgccaac tacaacattg gataccaggg cttctacgtt |
| 2341 | cctgagggtt acaaggatcg catgtactcc ttcttcagaa acttccagcc catgagtaga |
| 2401 | caggtggttg atgagattaa ctacaaagac tataaagctg tcgccgtacc ctaccagcat |
| 2461 | aataactctg gctttgtggg ttacatggct cctaccatgc gtcagggtca agcgtaccct |
| 2521 | gctaactacc cataccccct aattggaacc actgcagtaa ccagtgtcac ccagaaaaaa |
| 2581 | ttcctgtgcg acaggaccat gtggcgcatc ccattctcta gcaacttcat gtccatgggt |
| 2641 | gcccttacag acctgggaca gaacttgctg tatgccaact cggcccatgc gctggacatg |
| 2701 | acttttgagg tggatcccat ggatgagccc accctgcttt atcttctttt cgaagtcttc |
| 2761 | gacgtggtca gagtgcacca gccacaccgc ggcgtcatcg aggccgtcta cctgcgcaca |
| 2821 | ccgttctccg ccggcaacgc caccacataa |

A set of serotype-specific oligonucleotide probes were designed to bind specifically to serotypes 3, 4, 14 and 21 as follows:

HAdV-3:
(SEQ ID NO: 6)
GTTAAAACCGATGACACTAATGG;
HAdV-4:
(SEQ ID NO: 7)
GGTGTGGGATTGACAGACACTTAC;
HAdV-14:
(SEQ ID NO: 9)
AGACCAAGCTTGGAAAGATGTAAAT;
and
HAdV-21:
(SEQ ID NO: 10)
GGGTGCAGATTGGAAAGAGC.

Probes were preferentially chosen to have a melting temperature, Tm, between about 50° C. and 56° C.

The HAdV-3 specific probe corresponds to nucleotides 2,616-2,638 of the 35,345 base pair genomic sequence of the HAdV-3 strain GB having the GenBank accession number AY599834 (version AY599834.1 GI:57115749), which 35,345 base pair sequence is hereby incorporated by reference.

The HAdV-4 specific probe corresponds to nucleotides 19,382-19,405 of the 35,964 base pair genomic sequence of the HAdV-4 strain NHRC 3 having the GenBank accession number AY599837 (version AY599837.1 GI:57115887), which 35,964 base pair sequence is hereby incorporated by reference.

The HAdV-14 specific probe corresponds to nucleotides 19,541-19,565 of the 34,764 base pair genomic sequence of the HAdV-14 strain de Wit having the GenBank accession number AY803294 (version AY803294.1 GI:57115621), which 34,764 base pair sequence is hereby incorporated by reference.

The HAdV-21 specific probe corresponds to nucleotides 1,299-1,318 of the 2,850 base pair, HAdV-21 hexon gene having the GenBank accession number AY008279 (version AY008279.1 GI:13919592), which 2,850 base pair sequence is hereby incorporated by reference.

In the case of HAdV-7, a separate PCR primer pair was required because in initial experiments using the HVR7′ primers, several probes designed for HAdV-7 showed cross reactivity with HAdV-3 (data not shown). To circumvent the undesired cross reactivity, a second set of primers were designed to amplify a 253 base pair amplicon from HAdV-7:

HAdV-7 Forward:
(SEQ ID NO: 4)
CGCCCAATACATCTCAGTGG;
and
HAdV-7 Reverse:
(SEQ ID NO: 5)
ACTCCAACTTGAGGCTCTGG.

The HAdV-7 forward primer and HAdV-7 reverse primer correspond to nucleotides 383-402 and 595-614, respectively of the 35,306 base pair genomic sequence of HAdV-7 strain Gomen having the GenBank accession number AY594255 (version AY593255.1 GI:51173294), which 35,306 base pair sequence is hereby incorporated by reference.

Based on the HAdV-7 primers, an oligonucleotide probe specific for HAdV-7 was designed having the sequence: GTGGATAGTTACAACGGGAGAAG (SEQ ID NO:8). The HAdV-7 probe showed no cross reactivity with HAdV-3 (or any of the other HAdv serotypes tested) and corresponds to nucleotides 399-421 of the 35,306 base pair genomic sequence of HAdV-7 strain Gomen having the GenBank accession number AY594255 (version AY594255.1 GI:51173294), which 35,306 base pair sequence is hereby incorporated by reference.

Thus, the first primer set amplifies the target sequences for serotypes 3, 4, 14, and 21 in the hexon gene and the second primer pair amplifies the target sequence for serotype 7 within a different region of the hexon gene.

The length, Tm, and GC content of the HAdV-serotype specific oligonucleotide probes are provided in Table 1.

TABLE 1
Properties of HAdV-3, -4, -7, -14, and -21
Oligonucleotide Probes.

| Sero- | | Tm | | |
|---|---|---|---|---|
| type | Length | (° C.) | GC | Sequence |
| HAdV-3 | 23 | 50.2 | 39.1% | GTTAAAACCGATGACACTAATGG |
| | | | | (SEQ ID NO: 6) |
| HAdV-4 | 24 | 54.1 | 50.0% | GGTGTGGGATTGACAGACACTT |
| | | | | AC |
| | | | | (SEQ ID NO: 7) |
| HAdV-7 | 23 | 51.5 | 47.8% | GTGGATAGTTACAACGGGAGAAG |
| | | | | (SEQ ID NO: 8) |
| HAdV-14 | 25 | 53.8 | 36.0% | AGACCAAGCTTGGAAAGATGTAA |
| | | | | AT |
| | | | | (SEQ ID NO: 9) |
| HAdV-21 | 20 | 53.6 | 55.0% | GGGTGCAGATTGGAAAGAGC |
| | | | | (SEQ ID NO: 10) |

PCR Amplification.

The multiplex PCR reaction was performed using the Multiplex PCR kit (Qiagen, Valencia, Calif.). The reaction contained 12.5 μl of 2× Master Mix Buffer, 2 pmol of each primer, Sigma Genosys, (The Woodlands, Tex.) (a total of 4 primers, i.e., HVR7′ Forward, HVR7′ Reverse, HAdV-7 Forward, and HAdV-7 Reverse), 5 μl of sample and 6.7 μl of water to produce a final volume of 25 μl. The resulting mixture was then thermo cycled using the following conditions: initial cycle at 95° C. for 15 min, followed by 35 cycles at 94° C. for 30 sec, 52° C. for 1.5 min, 72° C. for 1 min and a final incubation at 72° C. 10 min.

The PCR product was then treated with 3.125 μl of shrimp alkaline phosphatase and 2.5 μl of exonuclease at 37° C. for 30 min followed by 99° C. for 30 seconds to remove the remaining dNTPs and primers.

Target Specific Primer Elongation (TSPE).

The reaction contained 2 μl of 10× Qiagen PCR Buffer, 0.5 μl of 50 mM MgCl₂, 0.15 μl of 5 U/μl of Tsp polymerase (Invitrogen), 0.1 μl of 1 mM dATP (Invitrogen, Carlsbad, Calif.), 0.1 μl of 1 mM dGTP (Invitrogen), 0.1 μl of 1 mM dTTP (Invitrogen), 0.25 μl of 4 mM biotin-dCTP (Invitrogen), 0.125 μl of HAdV-3 specific probe, 0.125 μl (1 μM) of HAdV-4 specific probe, 0.125 μl (1 μM) of HAdV-7 specific probe, 0.125 μl (1 μM) of HAdV-14 specific probe, and 0.125 μl (1 μM) of HAdV-21 specific probe, 5 μl ExoSAP-IT® (USB, Cleveland, Ohio) treated PCR product and 11.2 μl of water to produce a final volume of 20 μl. The resulting mixture was then thermo cycled using the following conditions: initial cycle at 95° C. for 2 min, followed by 40 cycles at 94° C. for 30 sec, 55° C. for 1 mM, 74° C. for 2 min. In the TSPE reaction, the oligonucleotide probes function as primers, facilitating the addition of nucleotides to their 3′ end in the presence of a polymerase, resulting in the generation of elongation or TSPE products. As shown by this reaction, the HAdV oligonucleotide probes designed for this assay retain their serotype specificity under hybridizing conditions at least as stringent as 55° C. with 1.25 mM MgCl₂.

Hybridization and Luminex Analysis.

Biotinylated TSPE products were hybridized to a fluid micro-bead array in wells of a 96-well plate and were detected using a streptavidin-phycoerythrin conjugate. The microsphere mix consists of 5 microspheres, each containing a different fluorescent dye mix and each coupled to a unique capture oligonucleotide sequence complementary to a unique tag oligonucleotide sequence incorporated into each of the five probes. 12.5 μl of the TSPE product and 12.5 μl of water were mixed with 25 μl of the microsphere mix (2500 microspheres per set) and were incubated at 96° C. for 2 min and then 37° C. for 30 min. After hybridization the plate was centrifuged at 2,250×g for 3 min and the supernatant removed. 2 μg/ml of streptavidin-phycoerythrin in 1×Tm (0.1M Tris-HCL, pH 8.0, 0.2M NaCl, 0.08% Triton X-100) was added to each well. The plate was incubated at 37° C. for 15 min in the dark. TSPE products bound to the microspheres were analyzed on the Luminex 200 at 37° C. TSPE products bound to the microspheres were detected with a streptavidin-phycoerythrin conjugate, and signals produced for each bead were analyzed by the Luminex and expressed as MFI units. Any signal that was greater than 3 times the highest background MFI signal was considered a positive call.

Analytical Specificity and Sensitivity.

The specificity of the multiplexed assay was examined by testing in duplicate 31 different pathogens, in triplicate, including 18 different HAdV serotypes. With this assay design, we observed TSPE signals only in the presence of the corresponding serotype, without cross-reactivity between TSPEs. The results show that the assay was able to accurately detect and identify HAdVs 3, 4, 7, 7a, 14, and 21 (Table 2) without cross reactivity between the various serotypes. To further test the performance of the assay the presence of multiple targets were tested in combination (Table 2).

Once the assay specificity was determined, the limit of detection for each of the detected serotypes was determined. This was achieved by testing in duplicate 5, 10-fold serial dilutions of the cultures from the strains of serotypes 3, 4, 7, 14, and 21 that were previously titered. The TCID₅₀ from each dilution was calculated based on the titer from the original undiluted culture. The lowest dilutions in which the TSPEs were able to detect the presence of the viruses are shown in Table 2.

With this assay design, we observed TSPE signals only in the presence of the corresponding serotype, without cross-reactivity between TSPEs. The assay was further tested with a combination of two HAdV serotypes in a single reaction. The assay was able to identify the two serotypes present in the reaction (Table 2).

TABLE 2
Assay specificity was assessed by testing the pathogens indicated and determining the lowest dilution detected
out of serial dilutions done for each reference HAdVs strain tested (see the text for details).

| | TSE signal/Lowest TCID₅₀ detected by the assay | | | | | Luminex RVP |
|---|---|---|---|---|---|---|
| Viral pathogen | HAdV3 | HAdV4 | HAdV7 | HAdV14 | HAdV21 | Assay call |
| HAdV3 | +/5 × 10³ | − | − | − | − | Adenovirus |
| HAdV4 | − | +/9.5 × 10³ | − | − | − | Adenovirus |
| HAdV7 | − | | +/1.1 × 10⁴ | − | − | Adenovirus |
| HAdV7a | − | − | +/ND | − | − | Adenovirus |
| HAdV14 | − | − | − | +/5 × 10³ | − | Adenovirus |
| HAdV21 | − | − | − | − | +/4.6 × 10⁵ | Adenovirus |
| HAdV 3 + 7 | + | − | + | − | − | ND* |
| HAdV 4 + 14 + 21 | − | + | − | + | + | ND* |
| HAdV 3 + 4 + 7+ 14 + 21 | + | + | + | + | + | ND* |
| HAdV 1, 2, 5, 6, 9, 11, | − | − | − | − | − | ND* |
| 16, 17, 31, 34, 35 and 40 | | | | | | |
| Influenza A H1N1 | − | − | − | − | − | Influenza A, H1 |
| Influenza A H3N2 | − | − | − | − | − | Influenza A, H3 |
| Influenza B | − | − | − | − | − | Influenza B |
| Corona OC43 | − | − | − | − | − | Corona OC43 |
| Corona SARS | − | − | − | − | − | Corona SARS |
| Parainfluenza 2 | − | − | − | − | − | Para 2 |
| Parainfluenza 3 | − | − | − | − | − | Para 3 |
| RSV A | − | − | − | − | − | RSV A |
| RSV B | − | − | − | − | − | RSV B |
| Metapneumovirus | − | − | − | − | − | Metapneumovirus |
| Enterovirus | − | − | − | − | − | Entero/Rhinovirus |
| Rhinovirus | − | − | − | − | − | Entero/Rhinovirus |
| Blank | − | − | − | − | − | − |

The corresponding TCID₅₀ of the lowest dilution detected is indicated.
ND* samples were not tested on the Luminex RVP kit but rather the Argene, Adenovirus r-gene ™

Evaluation of Clinical Samples.

To evaluate the assay's performance using clinical specimens, we tested 104 respiratory samples known to contain HAdV. The serotype of the HAdV present in the samples was also known. These samples were tested blindly in three independent runs.

Based on the 104 clinical samples tested, the sensitivity for the different serotypes tested were 90%, 95%, 100%, 100% and 85% for Ad3, Ad4, Ad7, Ad14 and Ad21, respectively, as shown in Table 3.

TABLE 3
Comparison of the results obtained
at NHARC by PCR/microneutralization
with the Luminex HAdV assay.

| | PCR/Microneutralization call | | | | | | | | |
|---|---|---|---|---|---|---|---|---|---|
| | HAdV serotype | | | | | | | Sensi- | Speci- |
| | 3 | 4 | 7 | 14 | 21 | Neg^{a} | Total | tivity | ficity |
| HAdV 3 | 9 | | | | | | 9 | 90% | 100% |
| HAdV 4 | | 19 | | | | 1 | 20 | 95% | 98% |
| HAdV 7 | | | 10^{b} | | | | 10^{d} | 100% | 100% |
| HAdV 7 + 4 | | | 1^{b} | | | | | | |
| HAdV 14 | | | | 19^{c} | | | 19^{d} | 100% | 100% |
| HAdV 14 + 4 | | | | 1^{c} | | | | | |
| HAdV 21 | | | | | 17 | | 17 | 85% | 100% |
| Neg^{a} | 1 | | | | 3 | 25 | 29 | | |
| Total | 10 | 19 | 10^{d} | 19^{d} | 20 | 26 | 104^{d} | | |

^{a}HAdV-negative specimens.
^{b}One HAdV7 sample by was positive for both HAdV 4 and 7, while one
^{c}HAdV14 sample by was positive for both HAdV14 and 4.
^{d}Samples with two calls were counted only once in the total.

Analysis of Discrepant Specimens.

A total of five discrepancies, as compared to the original calls obtained by PCR/microneutralization at NHRC, were observed. Three HAdV-21 and one HAdV-3 samples were called as negative by our assay, while a negative specimen tested positive for HAdV-4 with our assay. These five discrepant specimens were tested by the Luminex RVP kit, and HAdV was detected in all except for one of the HAdV-21 specimens, suggesting at least one of the samples could be a real negative.

We observed that the oligonucleotide probe for serotype 21 showed the lowest sensitivity (4.6×10⁵ TCID₅₀), which may explain, at least in part, these false negatives. Notwithstanding these discrepancies, the results, when observed collectively, show that this multiplexed HAdV assay is useful as a diagnostic tool in clinical settings. In the case of serotype 21, it may be possible to further improve the HAdV-21 oligonucleotide probe, but the performance observed with the clinical specimens suggests it still has potential as a diagnostic tool.

Co-Infections.

In our study, two samples were found to be co-infected: one with HAdV-4/HAdV-14 and the other with HAdV-4/HAdV-7. Previously, these samples were determined to be infected with only one HAdV serotype (either HAdV-14 or HAdV-7) by standard PCR testing, indicating that the present assay has increased sensitivity over standard PCR testing. Previous work by Vora et al. (19) showed a higher rate of co-infections paired with HAdV-4 or HAdV-7. This is not surprising because they are the two most common ARD associated serotypes (19), and suggests that our multiplex assay accurately detected co-infected samples. Typically, co-infections are harder to detect because one serotype, usually the one with a higher titer, often dominates the reaction, especially when relying on immunological methods. However, our multiplex assay is capable of detecting co-infections and is sensitive enough to detect the non-dominant serotype. Co-infections are of interest because they provide the opportunity for adenovirus strains to recombine and possibly form new variants. The methods described herein have the ability to detect co-infections in a single assay.

Currently most molecular techniques available may detect multiple HAdV serotypes within a single reaction, and even identify the species to which the HAdV belongs, but they are not designed to identify the specific serotype of the HAdV detected and, thus, require an additional sequencing reaction, restriction enzyme reaction, and/or phylogenetic analysis to discriminate between HAdV serotypes. This assay not only has the ability to detect multiple serotypes, including those of clinical relevance, but can also identify which ones are present in a single reaction. When challenged with various other common respiratory viruses and adenovirus serotypes there was no cross reactivity detected.

With the reintroduction of the vaccine program in the military recruit population there are fears that different serotypes may emerge as the dominant culprits of ARD outbreak at the various base camps. The ability to quickly identify the shifts in serotype dominance enables a more informed assessment of the vaccine's efficacy. Past studies have indicated that there is an increase in levels of neutralizing antibodies in the serum against HAdV-3 and HAdV-14 after HAdV-7 immunization (10, 18). The methods described herein, thus, provide a simple assay for timely detection and identification of five of the most common HAdV serotypes found among military patients (HAdV-3, -4, -7, -14 and -21).

### Example 2

### Identification of HAdV-1, -2, -5, and -6

The assay described in Example 1 permits the identification of up to five HAdV in a single reaction and can be expanded to include additional serotypes of interest. For example, pediatric and immunocompromised patients tend to experience infections of Group C HAdV serotypes. A set of serotype-specific oligonucleotide probes have been designed to bind specifically to Group C serotypes, HAdV-1, HAdV-2, HAdV-5, and HAdV-6, as set forth in Table 4. These oligonucleotide probes were designed to work in conjunction with the primers used in Example 1. Preliminary work indicates that these oligonucleotide probes hybridize specifically to the serotype of interest (probe for HAdV exhibited some cross reactivity) and can be used in conjunction with the probes for HAdV-3, HAdV-4, HAdV-7, HAdV-14, and HAdV-21 to identify simultaneously the presence of one or more of HAdV-1, -2, -3, -4, -5, 6, -7, -14, and -21 in a single reaction. Alternatively, one could use the oligonucleotide probes specific for HAdV-1, HAdV-2, HAdV-5, and HAdV-6 in the methods described herein, using only the HVR7′ forward and HRV7′ reverse primers, to identify simultaneously one or more of HAdV-1, HAdV-2, HAdV-5, or HAdV-6 in a sample. The oligonucleotide probe for HAdV-1 showed some cross reactivity and is being optimized to eliminate the cross reactivity.

TABLE 4
Properties of HAdV-1, -2, -5, and -6 Oligo-
nucleotide Probes.

| | | Tm | | |
|---|---|---|---|---|
| Serotype | Length | (° C.) | GC | Sequence |
| HAdV-1 | 21 | 52.9 | 47.6% | CAAACGGAAACGGTAATCCTC |
| | | | | (SEQ ID NO: 11) |
| HAdV-2 | 20 | 54.7 | 55.0% | CTAATGGCAATGGCTCAGGC |
| | | | | (SEQ ID NO: 12) |
| HAdV-5 | 25 | 53.5 | 40.0% | AAGGTAAAACCTAAAACAGGT |
| | | | | CAGG |
| | | | | (SEQ ID NO: 13) |
| HAdV-6 | 19 | 54.6 | 63.2% | GCTGCTAACGGGGACCAAG |
| | | | | (SEQ ID NO: 14) |

Probes were preferentially chosen to have a melting temperature, Tm, between about 50° C. and 56° C.

The HAdV-1 specific probe corresponds to nucleotides 20,188 to 20,208 of the 36,001 base pair genomic sequence of the HAdV-1 strain having the GenBank accession number AF534906 (version AF534906.1 GI:33330439), which 36,001 base pair sequence is hereby incorporated by reference.

The HAdV-2 specific probe corresponds to nucleotides 8,369 to 8,388 of the 35,937 base pair genomic sequence of the HAdV-2 strain having the GenBank accession number AC_{—}000007 (version AC_{—}000007.1 GI:56160492), which 35,937 base pair sequence is hereby incorporated by reference.

The HAdV-5 specific probe corresponds to nucleotides 20,123 to 20,147 of the 35,938 base pair genomic sequence of the HAdV-5 strain having the GenBank accession number BK000408 (version BK000408.1 GI:33694637), which 35,938 base pair sequence is hereby incorporated by reference.

The HAdV-6 specific probe corresponds to nucleotides 1,321 to 1,339 of the 2892 base pair, HAdV-6 hexon gene having the GenBank accession number AB330087 (version AB330087.1 GI:190356534), which 2892 base pair sequence is hereby incorporated by reference.

In addition, a universal oligonucleotide probe was designed to hybridize specifically with the Group C serotypes, HAdV-1, -2, -5, and -6. The sequence of this universal probe is AACAAGCGAGTGGTGGCTC (SEQ ID NO:15).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### REFERENCES

The following references are cited in the application and provide general information on the field of the invention and provide assays and other details discussed in the application. The following references are incorporated herein by reference in their entirety.
1. Adhikary, A. K., T. Inada, U. Banik, J. Numaga, and N. Okabe. 2004. Identification of subgenus C adenoviruses by fiber-based multiplex PCR. J Clin Microbiol 42:670-3.
2. Adrian, T., G. Wadell, J. C. Hierholzer, and R. Wigand. 1986. DNA restriction analysis of adenovirus prototypes 1 to 41. Arch Virol 91:277-90.
3. Blasiole, D. A., D. Metzgar, L. T. Daum, M. A. Ryan, J. Wu, C. Wills, C. T. Le, N. E. Freed, C. J. Hansen, G. C. Gray, and K. L. Russell. 2004. Molecular analysis of adenovirus isolates from vaccinated and unvaccinated young adults. J Clin Microbiol 42:1686-93.
4. Chmielewicz, B., A. Nitsche, B. Schweiger, and H. Ellerbrok. 2005. Development of a PCR-based assay for detection, quantification, and genotyping of human adenoviruses. Clin Chem 51:1365-73.
5. Damen, M., R. Minnaar, P. Glasius, A. van der Ham, G. Koen, P. Wertheim, and M. Beld. 2008. Real-time PCR with an internal control for detection of all known human adenovirus serotypes. J Clin Microbiol 46:3997-4003.
6. Gerna, G., E. Cattaneo, M. G. Revello, and M. Battaglia. 1982. Grouping of human adenoviruses by early antigen reactivity. J Infect Dis 145:678-82.
7. Gu, Z., S. W. Belzer, C. S. Gibson, M. J. Bankowski, and R. T. Hayden. 2003. Multiplexed, real-time PCR for quantitative detection of human adenovirus. J Clin Microbiol 41:4636-41.
8. Hierholzer, J. 1995. Adenoviruses, p. 169-188. In E. H. Lennette, D. A. Lennette, and E. T. Lennette (ed.), Diagnostics procedures for viral, rickettsial and chlamydial infections, 7th ed. American Public Health Association, Washington, D.C.
9. Malasig, M. D., P. R. Goswami, L. K. Crawford-Miksza, D. P. Schnurr, and G. C. Gray. 2001. Simplified microneutralization test for serotyping adenovirus isolates. J Clin Microbiol 39:2984-6.
10. Metzgar, D., M. Osuna, A. E. Kajon, A. W. Hawksworth, M. Irvine, and K. L. Russell. 2007. Abrupt emergence of diverse species B adenoviruses at US military recruit training centers. J Infect Dis 196:1465-73.
11. Metzgar, D., M. Osuna, S. Yingst, M. Rakha, K. Earhart, D. Elyan, H. Esmat, M. D. Saad, A. Kajon, J. Wu, G. C. Gray, M. A. Ryan, and K. L. Russell. 2005. PCR analysis of egyptian respiratory adenovirus isolates, including identification of species, serotypes, and coinfections. J Clin Microbiol 43:5743-52.
12. Miura-Ochiai, R., Y. Shimada, T. Konno, S. Yamazaki, K. Aoki, S. Ohno, E. Suzuki, and H. Ishiko. 2007. Quantitative detection and rapid identification of human adenoviruses. J Clin Microbiol 45:958-67.
13. Pabbaraju, K., K. Tokaryk, S. Wong, and J. Fox. 2008. Comparison of the Luminex xTAG Respiratory Viral Panel with In-House Nucleic Acid Amplification Tests for Diagnosis of Respiratory Virus Infections. Journal Of Clinical Microbiology 46:3056-3062.
14. Rubin, B. A. 1993. Clinical picture and epidemiology of adenovirus infections (a review). Acta Microbiol Hung 40:303-23.
15. Ryan, M. A., G. C. Gray, B. Smith, J. A. McKeehan, A. W. Hawksworth, and M. D. Malasig. 2002. Large epidemic of respiratory illness due to adenovirus types 7 and 3 in healthy young adults. Clin Infect Dis 34:577-82.
16. Sarantis, H., G. Johnson, M. Brown, M. Petrie, and R. Tellier. 2004. Comprehensive detection and serotyping of human adenoviruses by PCR and sequencing. J Clin Microbiol 42:3963-9.
17. Schmitz, H., R. Wigand, and W. Heinrich. 1983. Worldwide epidemiology of human adenovirus infections. Am J Epidemiol 117:455-66.
18, van der, V., and A. Prins. 1960 Studies of the significance of the recall phenomenon in the antibody response to adenovirus vaccine and infection. J Immunol 84:562-8.
19. Vora, G. J., B. Lin, K. Gratwick, C. Meador, C. Hansen, C. Tibbetts, D. A. Stenger, M. Irvine, D. Seto, A. Purkayastha, N. E. Freed, M. G. Gibson, K. Russell, and D. Metzgar. 2006. Co-infections of adenovirus species in previously vaccinated patients. Emerg Infect Dis 12:921-30.
20. Wong, S., K. Pabbaraju, X. L. Pang, B. E. Lee, and J. D. Fox. 2008. Detection of a broad range of human adenoviruses in respiratory tract samples using a sensitive multiplex real-time PCR assay. J Med Virol 80:856-65.
21. Wood, S. R., I. R. Sharp, E. O. Caul, I. Paul, A. S. Bailey, M. Hawkins, S. Pugh, J. Treharne, and S. Stevenson. 1997. Rapid detection and serotyping of adenovirus by direct immunofluorescence. J Med Virol 51:198-201.
22. Xu, W., and D. D. Erdman. 2001. Type-specific identification of human adenovirus 3, 7, and 21 by a multiplex PCR assay. J Med Virol 64:537-42.
23. Xu, W., M. C. McDonough, and D. D. Erdman. 2000. Species-specific identification of human adenoviruses by a multiplex PCR assay. J Clin Microbiology 38:4114-4120.
24. Davison et al., Maria Benkö and Balázs Harrach. 2003. Genetic content and evolution of adenoviruses. J Gen Vir 84:2895-908.
25. Ebner, K., W. Pinsker, and T. Lion. 2005. Comparative sequence analysis of the hexon 15 gene in the entire spectrum of human adenovirus serotypes: phylogenetic, taxonomic, and 16 clinical implications. J Virol 79:12635-42.
26. Houng, H. S., S. Clavio, K. Graham, R. Kuschner, W. Sun, K. L. Russell, and L. N. Binn. 2006. Emergence of a new human adenovirus type 4 (Ad4) genotype: identification of a novel inverted terminal repeated (ITR) sequence from majority of Ad4 isolates from US military recruits. J Clin Virol 35:381-7.
27. Sanchez, J. L., L. N. Binn, B. L. Innis, R. D. Reynolds, T. Lee, F. Mitchell-Raymundo, S. C. Craig, J. P. Marquez, G. A. Shepherd, C. S. Polyak, J. Conolly, and K. F. Kohlhase. 2001. Epidemic of adenovirus-induced respiratory illness among US military recruits: epidemiologic and immunologic risk factors in healthy, young adults. J Med Virol 65:710-8.

1. A method of determining whether a sample contains one or more of human adenovirus-3 (HAdV-3), HAdV-4, HAdV-7, HAdV-14, and HAdV-21, wherein the sample comprises nucleic acid, the method comprising:
a) amplifying the nucleic acid in the sample using a first pair of primers and a second pair of primers, wherein the first pair of primers are designed to amplify a first region of a human adenovirus hexon gene and the second pair of primers are designed to amplify a second region of the human adenovirus hexon gene, and wherein if at least one of HAdV-3, HAdV-4, HAdV-14, or HAdV-21 is present in the test sample, a first amplification product is produced and if HAdV-7 is present in the sample a second amplification product is produced;
b) incubating any first or second amplification product produced during the amplification step under hybridizing conditions with a first oligonucleotide probe, a second oligonucleotide probe, a third oligonucleotide probe, a fourth oligonucleotide probe, and a fifth oligonucleotide probe, wherein the first oligonucleotide probe comprises a first unique tag sequence and is specific for HAdV-3, the second oligonucleotide probe comprises a second unique tag sequence and is specific for HAdV-4, the third oligonucleotide probe comprises a third unique tag sequence and is specific for HAdV-7, the fourth oligonucleotide probe comprises a fourth unique tag sequence and is specific for HAdV-14, and the fifth oligonucleotide probe comprises a fifth unique tag sequence and is specific for HAdV-21;
c) elongating any oligonucleotide probe hybridized to the first or second amplification product in the presence of a polymerase and four deoxyribonucleotide triphosphates to form one or more elongation products, wherein at least one of the deoxyribonucleotide triphosphates comprises a first label;
d) separating the elongation products from the first or second amplification product under denaturing conditions;
e) incubating the elongation products with a solid support under hybridizing conditions, wherein the solid support comprises a first unique capture oligonucleotide having a recognition sequence that is complementary to the first unique tag sequence in the first oligonucleotide probe, a second unique capture oligonucleotide having a recognition sequence that is complementary to the second unique tag sequence in the second oligonucleotide probe, a third unique capture oligonucleotide having a recognition sequence that is complementary to the third unique tag sequence in the third oligonucleotide probe, a fourth unique capture oligonucleotide having a recognition sequence that is complementary to the fourth unique tag sequence in the fourth oligonucleotide probe, and a fifth unique capture oligonucleotide having a recognition sequence that is complementary to the fifth unique tag sequence in the fifth oligonucleotide probe; and
f) analyzing the solid support to determine if the sample contains one or more of the HAdV-3 serotype, the HAdV-4 serotype, the HAdV-7 serotype, the HAdV-14 serotype, or the HAdV-21 serotype,
wherein if the sample contains HAdV-3, the first unique capture oligonucleotide hybridizes with the first unique tag sequence in the one or more elongation products, thereby indicating the presence of the HAdV-3 serotype in the sample;
wherein if the sample contains HAdV-4, the second unique capture oligonucleotide hybridizes with the second unique tag sequence in the one or more elongation products, thereby indicating the presence of the HAdV-4 serotype in the sample;
wherein if the sample contains HAdV-7, the third unique capture oligonucleotide hybridizes with the third unique tag sequence in the one or more elongation products, thereby indicating the presence of the HAdV-7 serotype in the sample;
wherein if the sample contains HAdV-14, the fourth unique capture oligonucleotide hybridizes with the fourth unique tag sequence in the one or more elongation products, thereby indicating the presence of the HAdV-14 serotype in the sample; and
wherein if the sample contains HAdV-21, the fifth unique capture oligonucleotide hybridizes with the fifth unique tag sequence in the one or more elongation products, thereby indicating the presence of the HAdV-21 serotype in the sample.

2. The method of claim 1, wherein the solid support comprises an array of microspheres, wherein the array of microspheres comprises a first microsphere comprising the first unique capture oligonucleotide, a second microsphere comprising the second unique capture oligonucleotide, a third microsphere comprising the third unique capture oligonucleotide, a fourth microsphere comprising the fourth unique capture oligonucleotide, and a fifth microsphere comprising the fifth unique capture oligonucleotide, and wherein each of the first, second, third, fourth, and fifth microspheres comprises a different fluorochrome or fluorescent dye.

3. The method of claim 1, wherein the first label is biotin and wherein the method further comprises after incubating the elongation product with the solid support, adding avidin or streptavidin, wherein the avidin or streptavidin comprises a second label.

4. The method of claim 3, wherein the second label is a fluorescent dye.

5. The method of claim 1, wherein in the detection step, the array of microspheres is analyzed by flow cytometry to determine if the sample contains one or more of the HAdV-3 serotype, the HAdV-4 serotype, the HAdV-7 serotype, the HAdV-14 serotype, or the HAdV-21 serotype.

6. The method of claim 1, wherein the first region of the human adenovirus hexon gene corresponds to about nucleotides 1003 to 1604 of SEQ ID NO:1 and the second region of the human adenovirus hexon gene corresponds to about nucleotides 383 to 614 of SEQ ID NO:1.

7. A kit for identifying one or more HAdV-3, HAdV-4, HAdV-7, HAdV-14, and HAdV-21 in a sample, wherein the kit comprises:
a) a first and second pair of primers, wherein the first pair of primers are designed to amplify a first region of a human adenovirus hexon gene and the second pair of primers are designed to amplify a second region of the human adenovirus hexon gene, and
b) a first, second, third, fourth, and fifth oligonucleotide probe, wherein the first oligonucleotide probe is specific for HAdV-3, the second oligonucleotide probe is specific for HAdV-4, the third oligonucleotide probe is specific for HAdV-7, the fourth oligonucleotide probe is specific for HAdV-14, and the fifth oligonucleotide probe is specific for HAdV-21.

8. The kit of claim 7, wherein the first region of the human adenovirus hexon gene corresponds to about nucleotides 1003 to 1604 of SEQ ID NO:1 and the second region of the human adenovirus hexon gene corresponds to about nucleotides 383 to 614 of SEQ ID NO:1.

9. The kit of claim 7, wherein the nucleotide sequences of the first pair of primers are CTGATGTACTACAACAGCACTGGCAACATGGG (SEQ ID NO:2) and CGGTGGTGGTTAAATGGATTCACATTGTCC (SEQ ID NO:3), and wherein the nucleotide sequences of the second set of primers are CGCCCAATACATCTCAGTGG (SEQ ID NO:4) and ACTCCAACTTGAGGCTCTGG (SEQ ID NO:5).

10. The kit of claim 7, wherein each of the first, second, third, fourth, and fifth oligonucleotide probes has about 20-25 nucleotides, a G/C content of at least about 36%, and a melting temperature between about 50° C. and 56° C.

11. The kit of claim 7, wherein the first oligonucleotide probe hybridizes under stringent conditions to the complement of a region of the HAdV-3 hexon gene corresponding to nucleotides 2,616 to 2,638 of the hexon gene of HAdV-3 having GenBank accession no. AY599834 (version AY599834.1 GI:57115749), the second oligonucleotide probe hybridizes under stringent conditions to the complement of a region of the HAdV-4 hexon gene corresponding to nucleotides 19,382 to 19,405 of the hexon gene of HAdV-4 of GenBank accession no. AY599837 (version AY599837.1 GI:57115887), the third oligonucleotide probe hybridizes under stringent conditions to the complement of a region of the HAdV-7 hexon gene corresponding to nucleotides 399 to 421 of the hexon gene of HAdV-7 of GenBank accession no. AY594255 (version AY594255.1 GI:51173294), the fourth oligonucleotide probe hybridizes under stringent conditions to the complement of a region of the HAdV-14 hexon gene corresponding to nucleotides 19,541 to 19,565 of the hexon gene of HAdV-14 of GenBank accession no. AY803294 (version AY803294.1 GI:57115621), and the fifth oligonucleotide probe hybridizes under stringent conditions to the complement of a region of the HAdV-21 hexon gene corresponding to nucleotides 1,299 to 1,318 of the hexon gene of HAdV-21 of GenBank accession no. AY008279 (version AY008279.1 GI:13919592).

12. The kit of claim 7, wherein the nucleotide sequence of the first oligonucleotide probe is GTTAAAACCGATGACACTAATGG (SEQ ID NO:6), the nucleotide sequence of the second oligonucleotide probe is GGTGTGGGATTGACAGACACTTAC (SEQ ID NO:7), the nucleotide sequence of the third oligonucleotide probe is GTGGATAGTTACAACGGGAGAAG (SEQ ID NO:8), the nucleotide sequence of the fourth oligonucleotide probe is AGACCAAGCTTGGAAAGATGTAAAT (SEQ ID NO:9), and the nucleotide sequence of the fifth oligonucleotide is GGGTGCAGATTGGAAAGAGC (SEQ ID NO:10).

13. An isolated oligonucleotide of about 20 to 25 nucleotides in length, wherein the nucleotide sequence of the oligonucleotide is selected from:
(SEQ ID NO: 6)
(a) GTTAAAACCGATGACACTAATGG,
(SEQ ID NO: 7)
(b) GGTGTGGGATTGACAGACACTTAC,
(SEQ ID NO: 8)
(c) GTGGATAGTTACAACGGGAGAAG,
(SEQ ID NO: 9)
(d) AGACCAAGCTTGGAAAGATGTAAAT,
(SEQ ID NO: 10)
(e) GGGTGCAGATTGGAAAGAGC,
(SEQ ID NO: 4)
(f) CGCCCAATACATCTCAGTGG,
(SEQ ID NO: 5)
(g) ACTCCAACTTGAGGCTCTGG,
or
(f) the complement of any one of (a), (b), (c),
(d), or (e).

14. The isolated oligonucleotide of claim 13, further comprising a label.

15. The isolated oligonucleotide of claim 13, wherein the nucleotide sequence of the oligonucleotide is:
(SEQ ID NO: 4)
(a) CGCCCAATACATCTCAGTGG,
or
(SEQ ID NO: 5)
(b) ACTCCAACTTGAGGCTCTGG.
